(19) United States
(12) Patent Application Publication
(10) Pub. No.: US 2024/0002465 A1
(43) Pub. Date: Jan. 4, 2024

(54) MR1 RESTRICTED T CELL RECEPTORS FOR CANCER IMMUNOTHERAPY

(71) Applicant: UNIVERSITÄT BASEL, Basel (CH)

(72) Inventors: Gennaro DE LIBERO, Bottmingen (CH); Marco LEPORE, Abingdon (GB); Lucia MORI, Bottmingen (CH)

(73) Assignee: UNIVERSITÄT BASEL, Basel (CH)

(21) Appl. No.: 18/353,119

(22) Filed: Jul. 17, 2023

Related U.S. Application Data
(63) Continuation-in-part of application No. 16/563,987, filed on Sep. 9, 2019, now Pat. No. 11,702,459.
Application No. 16/563,987 is a continuation-in-part of application No. PCT/EP2018/055620, filed on Mar. 7, 2018.

(30) Foreign Application Priority Data
Mar. 7, 2017 (EP) 17159754.5
Jul. 3, 2017 (EP) 17179309.4
Sep. 12, 2018 (EP) 18194025.5

(51) Int. Cl. C07K 14/725; C12N 15/86

(52) U.S. Cl. CPC C07K 14/7051; C12N 15/86

(57) ABSTRACT

Described herein is a method of isolating a T cell that expresses a T cell receptor capable of binding specifically to an antigen presented by a cancer cell in association with an MR1 molecule. The method comprises the steps of (a) providing a preparation of T cells, (b) contacting the preparation with cancer cells expressing MR1 protein; (c) isolating a T cell that is specifically reactive to said cancer cells. The invention further relates to a method of preparing a T cell preparation expressing select MR1 recognizing T cell receptors from transgene expression vectors, the use of such T cell preparations in treatment of cancer, and to collections of MR1 reactive T cell receptor encoding nucleic acids and cells.

### CROSS-REFERENCE TO RELATED APPLICATIONS

This is a continuation-in-part of U.S. patent application Ser. No. 16/563,987, filed Sep. 9, 2019, which is a continuation-in-part of International Patent Application No. PCT/EP2018/055620 filed Mar. 7, 2018, which claims the benefit of European Patent Application Nos. EP 17159754.5 filed Mar. 7, 2017 and EP17179309.4 filed Jul. 3, 2017. Benefit is also claimed to European Patent Application No. EP 18194025.5, filed Sep. 12, 2018. The foregoing patent applications are incorporated by reference herein in their entirety.

### FIELD

The invention relates to the identification of tumour-reactive human T cell antigen receptors (TCRs) restricted to the non-polymorphic antigen-presenting molecule MR1. The functional TCR transcript sequences were isolated from clones representative of a novel population of human T cells (discovered by the inventors and termed MR1T cells) reacting to MR1-expressing tumour cells in the absence of any added foreign antigen and in MR1-dependent manner. The invention also relates to the use of MR1-restricted tumour-reactive TCR gene sequences in cancer treatment.

### BACKGROUND OF THE INVENTION

T lymphocytes can detect a diverse range of non-peptide antigens including lipids and phosphorylated isoprenoids, presented by non-polymorphic cell surface molecules. The heterogeneous phenotypic and functional properties of these T cells support specialized roles in host protection against infections, autoimmunity, and cancer. The repertoire of T cells specific for non-peptide antigens recently increased to include mucosal associated invariant T (MAIT) cells, which respond to small riboflavin precursors produced by a wide range of yeasts and bacteria, and presented by the MHC class I-related protein MR1. MAIT cells are frequent in human blood, kidney and intestine, and comprise a major fraction of T cells resident in the liver. Following activation, MAIT cells release an array of pro-inflammatory and immunomodulatory cytokines, and can mediate direct killing of microbe-infected cells. It remains unknown whether the role of MR1 extends beyond presentation of microbial metabolites to MAIT cells.

MR1 is a non-polymorphic MHC class I-like protein that is expressed at low levels on the surface of many cell types. MR1 is highly conserved across multiple species, with human and mouse MR1 sharing >90% sequence homology at the protein level.

The inventors proposed the existence of human T cells that recognize tumour-associated antigens presented by MR1. These novel T cells might participate in tumour immune surveillance, thus representing novel tools for cancer immunotherapy. Adoptive therapy with donor- or patient-derived T cells engineered to express TCRs specific for selected tumour-associated antigens represents a promising and safe strategy to induce clinically relevant anti-tumour immune response in cancer patients. Nevertheless, the majority of the so far identified tumour-associated antigens are peptides presented by polymorphic MHC molecules. The extreme polymorphism of MHC genes limits the application of this approach to those patients expressing unique MHC alleles. Targeting tumour-antigens bound to non-polymorphic antigen presenting molecules, such as MR1, might overcome this constraint and in principle be applicable to all patients bearing tumours expressing MR1. The use of tumour-reactive T cell receptors that recognize MR1-presented antigens might also have the advantage of complementing anti-tumour responses mediated by MHC-presented peptide antigens, excluding cross-competition of tumour antigens for binding to the same type of presenting molecule. In addition, this strategy may provide the possibility of targeting antigens of different nature on the same tumour cells, thus minimizing the potential occurrence of tumour escape variants under selective immune pressure. Therefore, the identification of MR1-presented tumour-associated antigens and the characterization of MR1-restricted TCRs recognizing these antigens might have important implications for cancer immunotherapy.

Based on this state of the art, the objective of the present invention is to provide novel means and methods of treatment for cancer. This objective is attained by the subject matter of the independent claims, with further advantageous solutions provided by the dependent claims, examples and figures disclosed herein.

### SUMMARY OF THE INVENTION

In the broadest sense, the invention relates to a method of treatment of cancer, wherein TCR sequences isolated from T cells reactive to MR1-expressing cancer cells (MR1T cells) are expressed after gene transfer into a population of a patient's T cells. These foreign, transgenically expressed TCR sequences are used for conferring specific recognition of MR1-expressing cancer cells to T cells as a treatment of the patient's tumour.

The invention similarly provides a T cell, and T cell preparations comprising a plurality of T cells, transduced with MR1T cell specific TCR genes. In certain embodiments, the T cells transduced with MR1T cell TCR genes can be used for adoptive cell immunotherapy in combination with other therapeutic interventions.

The invention also relates to a research method facilitating the identification of the TCR sequences isolated from T cells reactive to MR1-expressing cancer cells (MR1T cells) employed in the invention. This encompasses a method to isolate MR1-restricted T cells that recognize tumour-associated antigens. T cells from peripheral blood of normal donors or from cancer patients are stimulated with tumor cell lines representing the same type of tumor in patient. These tumor cell lines are transfected with the MR1 gene and thus express large amounts of MR1 protein on their plasma membrane. Activated T cells are sorted for expression of activation markers, (e.g. CD137, or CD150, or CD69, or ICOS) and are cloned as published (De Libero, Methods for the generation of T cell clones and epithelial cell lines from excised human biopsies or needle aspirates. In MHC 123-140 (IRL, Oxford; 1997)). Individual clones are tested for their capacity to recognize tumor cells in an MR1-restricted manner, for killing tumor cells and for release of inflammatory cytokines. The TCR genes of MR1-restricted and tumor-specific T cell clones are sequenced and identified.

The invention also relates to a method by which tumor-infiltrating T cells are prepared from the same cancer tissue biopsies according to our previously established protocol (De Libero, ibid.). Individual T cell clones are tested against a panel of tumor cell lines expressing MR1 protein. The most reactive T cell clones are studied for their MR1 restriction, tumor killing and release of inflammatory cytokines. The TCR genes of selected T cell clones are sequenced.

### BRIEF DESCRIPTION OF THE FIGURES

FIGS. 1A-1E. MR1T cells do not recognize microbial antigens. (FIG. 1A) Surface expression of MR1 by CCRFSB, THP-1 and A375-MR1 cells. Grey histograms indicate staining with isotype-matched control mAbs. Stimulation of (FIG. 1B) MR1T cell clone DGB129 or (FIG. 1C) MAIT cell clone SMC3 by the three cell lines in A in the absence (no Ag) or presence of E. coli lysate (E. coli) and/or anti-MR1 blocking mAbs (α-MR1). The MAIT clone SMC3 was previously isolated from PBMC of a healthy donor and expresses canonical MAIT phenotype and function. Columns indicate IFN-γ release (mean+SD). Stimulation of (FIG. 1D) DGB129 MR1T or (FIG. 1E) SMC3 MAIT cells by THP-1 cells, constitutively expressing surface MR1, loaded with synthetic 6,7-dimethyl-8-D-ribityllumazine (RL-6,7-diMe) with or without anti-MR1 mAbs. Columns indicate mean IFN-γ release+SD. Data are representative of four (A, B and C), two (D and E). *P<0.05 (Unpaired Student's t-test).

FIGS. 2A-2E. Isolation strategy of MR1T cell clones from peripheral T cells. (FIG. 2A) FACS analysis of purified T cells previously expanded with irradiated A375-MR1 cells following overnight co-culture with A375-MR1 cells in the absence of exogenous antigens. Left dot plot shows CD3 and CellTrace violet (CTV) staining in live cells. Right dot plot shows CD69 and CD137 expression of CD3-positive CTV-negative gated cells. Arrows indicate gating hierarchy. Numbers indicate the percentages of cells within the gates. Cells from Donor A are illustrated as a representative donor. (FIG. 2B, FIG. 2D) Cumulative results of T cell clones screening from Donors A and B. T cell clones were generated from CD3⁺CTV⁻CD137⁺ sorted T cells as depicted in A. Graphs show the individual clones (x axis) and their IFN-γ release (y axis), expressed as ratio between the amount of cytokine secreted in response to A375-MR1 cells vs. A375 WT cells. Each dot represents a single T cell clone, tested at the same time in the indicated experimental conditions. The vertical lines indicate the number of T cell clones displaying MR1-restricted reactivity (i.e. the clones showing an IFN-γ release ratio above the arbitrary cut-off of 2). Results are representative of two independent experiments. (FIG. 2C, FIG. 2E) IFN-γ release by 14 representative clones from Donor A and 11 clones from Donor B after stimulation with A375 WT, A375-MR1 and A375-MR1 in the presence of blocking anti-MR1 mAbs (α-MR1). Dots represent the IFN-γ release (mean±SD of duplicate cultures) by each clone. Results are representative of three independent experiments. *P<0.05 (Unpaired Student's t-test).

FIGS. 3A-3D. MR1T cells are common in the blood of healthy individuals. (FIG. 3A) Flow cytometry analysis of purified T cells from a representative donor (Donor C) after overnight co-culture with A375 WT or A375-MR1 cells. Dot plots show CD69 and CD137 expression on live CD3⁺ cells. Numbers indicate the percentage of cells in the gates. (FIG. 3B) Frequency of CD69⁺CD137⁺ T cells from 5 different donors after overnight co-culture with A375 WT or A375-MR1 cells. (FIG. 3C) Cumulative results of T cell clone stimulation assays from Donor C. T cell clones were generated from CD3⁺CD69⁺CD137⁺ sorted T cells as depicted in A, right dot plot. The graph shows the number of tested clones (x axis) and IFN-γ release (y axis) expressed as ratio between the amounts of cytokine secreted in response to A375-MR1 cells vs. A375 WT cells. Each dot represents a single T cell clone, tested at the same time in the indicated experimental conditions. The vertical line indicates the number of T cell clones displaying MR1-restricted reactivity (i.e. the clones showing an IFN-γ release ratio above arbitrary cut-off of 2). Results are representative of two independent experiments. (FIG. 3D) Recognition of A375-MR1 but not A375 WT cells in the absence of exogenous antigens by 8 representative MR1-restricted T cell clones from Donor C. Inhibition of T cell clone reactivity to A375-MR1 cells by blocking anti-MR1 mAbs (α-MR1). Dots represent the IFN-γ release (mean±SD of duplicate cultures) by each clone tested in the three experimental conditions. Results are representative of three independent experiments. *P<0.05 (Unpaired Student's t-test).

FIGS. 4A-4E. MR1T TCR gene transfer confers MR1-restricted recognition of A375 cells. Stimulation of (FIG. 4A) SKW-3 cells expressing the DGB129 TCR (SKW3-DGB129) and (FIG. 4B) J.RT3-T3.5 cells expressing the MAIT MRC25 TCR (J.RT3-MAIT) with A375 cells that expressed (A375-MR1) or lacked (A375 WT) MR1, with or without E. coli lysate and anti-MR1 mAbs. Stimulation of SKW-3 cells expressing the TCRs of three individual MR1T cell clones (FIG. 4C) DGA4 (SKW3-DGA4), (FIG. 4D) DGB70 (SKW3-DGB70) and (FIG. 4E) JMA (SKW3-JMA) with A375-MR1 or A375 WT cells in the presence or not of or anti-MR1 mAbs. CD69 median fluorescence intensity (MFI)+SD of duplicate cultures of transduced T cells are shown. The CD69 MFI of transduced T cells cultured in the absence of APCs is also shown. Mock-transduced T cells showed background levels of CD69 expression when incubated with A375-MR1 or A375 WT (not shown). Data are representative of three independent experiments. *P<0.05 (Unpaired Student's t-test).

FIGS. 5A-5B. Differential recognition of various types of tumour cells by MR1T cell clones. (FIG. 5A) Recognition of four human cells lines expressing constitutive surface levels of MR1 by the representative SMC3 MAIT cell clone in the absence (no Ag) or presence of E. coli lysate (E. coli) with or without anti-MR1 blocking mAbs (α-MR1). (FIG. 5B) Recognition of the same cell types as in A by thirteen MR1T cell clones with or without anti-MR1 mAbs (α-MR1). Graphs show IFN-γ release (mean±SD of duplicate cultures).

FIGS. 6A-6C. MR1T cell clones do not react to microbial ligands or to 6-FP. (FIG. 6A) Response of seven MR1T cell clones and one control MAIT cell clone co-cultured with A375 cells expressing (A375-MR1) or not (A375 WT) MR1 in the presence or absence of E. coli lysate. Blocking of T cell clone reactivity by anti-MR1 mAbs (α-MR1) is also shown. (FIG. 6B) Response of MR1T cell clones to A375 cells expressing either WT MR1 molecules (A375-MR1) or K43A-mutated MR1 molecules (A375-MR1 K43A) in the presence of 6-formyl pterin (6-FP). (FIG. 6C) Stimulation of control MAIT cell clone MRC25 or control TCR Vγ9Vδ2 clone G2B9 with A375-MR1 or A375-MR1 K43A cells previously incubated with or without E. coli lysate or zoledronate, respectively, either in the absence or presence of 6-FP. Results are expressed as mean±SD of IFN-γ measured in duplicate cultures. Results are representative of three independent experiments. *P<0.05 (Unpaired Student's t-test).

FIGS. 7A-7E. MR1T cell clones do not recognize Ac-6-FP. (FIG. 7A) Stimulation of three representative MR1T cell clones by A375-MR1 cells in the absence or presence of acetyl-6-formyl pterin (Ac-6-FP). (FIG. 7B) Stimulation of two MAIT cell clones (MRC25 and SMC3) by A375-MR1 cells pulsed with E. coli lysate in the absence or presence of Ac-6-FP. (FIG. 7C) A375-MR1 cells were treated with zoledronate (Zol) in the absence or presence of Ac-6-FP (25 μg/ml) and used to stimulate a TCR Vγ9-Vδ2 cell clone (G2B9). (FIG. 7D) A375 cells expressing K43A mutant MR1 molecules (A375-MR1 K43A) were used to stimulate the three MR1T cell clones shown in A, in the absence or presence of Ac-6-FP (25 μg/ml). (FIG. 7E) Stimulation of the two MAIT cell clones used in B by A375-MR1 K43A cells pulsed with E. coli lysate in the absence or presence of Ac-6-FP (25 μg/ml). Results are expressed as mean±SD of IFN-γ release assessed in duplicate cultures and are representative of three independent experiments. *P<0.05 (Unpaired Student's t-test).

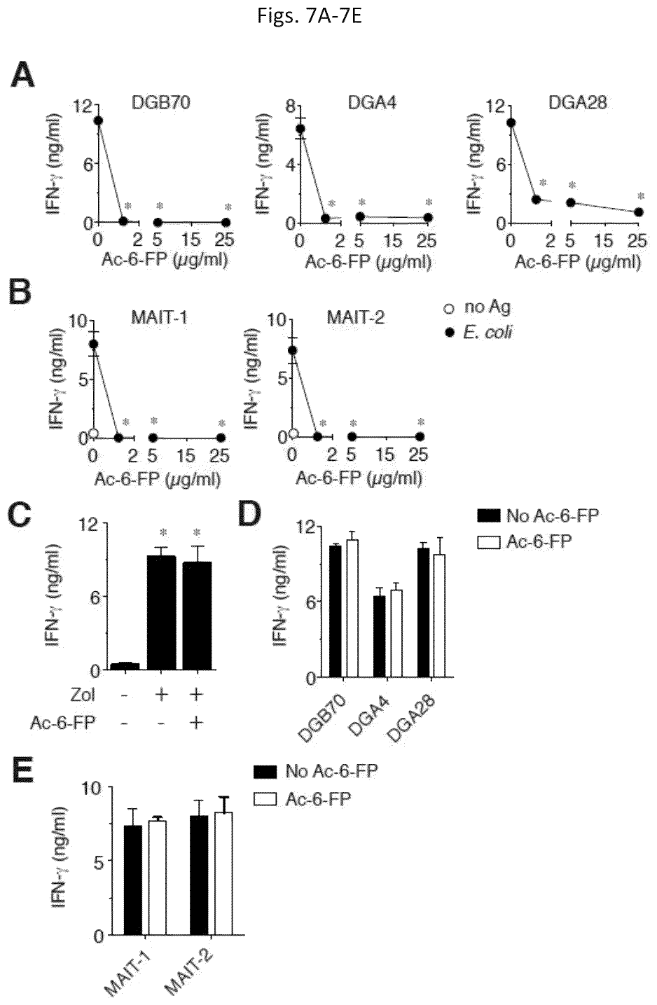

FIGS. 8A-8F. MR1T cells recognize antigens present in tumour cells and not derived from RPMI 1640 medium. Stimulation of the DGB129 MR1T cell clone by MR1-overexpressing (FIG. 8A) A375 cells (A375-MR1) and (FIG. 8B) THP-1 cells (THP1-MR1) grown for 4 days in RPMI 1640 or in PBS both supplemented with 5% human serum. Inhibition of T cell clone reactivity by anti-MR1 blocking mAbs (α-MR1) is shown. DGB129 cells recognize APCs loaded with fractions isolated from (FIG. 8C) THP-1 cell lysate or from (FIG. 8D) in vivo grown mouse breast tumour EMT6. Fractions E1 and E2 contain hydrophobic molecules; fractions N1-N4 contain hydrophilic molecules. (FIG. 8E) DGB70 MR1T cells react to N3 fraction of THP-1 lysate. (FIG. 8F) Stimulation of DGB129 and DGB70 T cells by THP-1-derived fractions N3 and N4 loaded onto plastic-bound recombinant MR1. Shown is T cell release of IFN-γ or GM-CSF mean±SD of duplicate cultures (representative of three independent experiments). Total cytokine release is shown in panels A, B, F; fold increase over background is shown in panels C, D, E. * P<0.05 (Unpaired Student's t-test).

FIGS. 9A-9F. MR1T cells display differential anti-tumour responses. The MR1-expressing tumour cell lines THP-1 and A375 were cultured overnight with the MR1T cell clones (FIG. 9A) DGB129 or (FIG. 9B) DGB70 at the indicated effector:target (E:T) ratios. The graphs show the percentages of apoptotic target cells in individual experimental conditions, assessed by flow cytometry using Annexin V and propidium iodide staining. MR1T cells were identified by staining with anti-CD3 mAbs and excluded from the analysis. Inhibition of MR1T cell clone killing capacity by anti-MR1 (α-MR1) mAbs is also shown at the 1:1 E:T ratio. (FIG. 9C) Recognition of Mo-DCs isolated from a healthy individual by thirteen MR1T cell clones with or without anti-MR1 mAbs (α-MR1). Graphs show IFN-γ release (mean±SD of duplicate cultures). (FIG. 9D) Recognition of Mo-DCs from three donors by the representative DGB129 MR1T cell clone in the absence or presence of anti-MR1 (α-MR1) mAbs. IFN-γ release in the supernatants is shown and expressed as mean±SD. (FIG. 9E) Flow cytometry analysis of co-stimulatory molecules CD83 and CD86 on Mo-DCs after co-culture with DGB129 MR1T cells with or without anti-MR1 mAbs (α-MR1). A control group consisting of Mo-DCs stimulated with LPS (10 ng/ml) in the absence of T cells is also shown. Numbers indicate percentages of cells in each quadrant. (FIG. 9F) Stimulation of JMAN MR1T cell clone by LS 174T and HCT116 gastrointestinal tumour cell lines and by normal gut epithelial cells (GEC) in the presence or not of anti-MR1 mAbs (α-MR1). Columns show IFN-γ release (mean±SD of duplicate cultures). All the results are representative of at least three independent experiments. P<0.05 (Unpaired Student's t-test).

FIGS. 10A-10B. Functional heterogeneity of MR1T cell clones. (FIG. 10A) IFN-γ released by 7 selected MR1T cell clones stimulated with A375-MR1 cells. ELISA results are expressed as mean±SD of IFN-γ release measured in duplicate cultures. (FIG. 10B) Analysis of 16 additional cytokines by multiplex cytokine assay performed on the same supernatants for which IFN-γ is shown in A. Results are representative of two independent experiments.

FIG. 11. MR1T cell clones display multiple chemokine-receptor expression profiles. Flow cytometry analysis of CXCR3, CCR4 and CCR6 surface expression by seven selected resting MR1T cell clones. Graphs show the relative fluorescence intensity calculated by dividing the median fluorescence intensity (MFI) of specific mAb staining by the MFI of the corresponding isotype control. Data are representative of two independent experiments.

FIG. 12. MR1T cells reduce the number of human melanoma lung nodules in mice. Immunocompromised NSG mice were injected with the human melanoma A375 cells expressing MR1 (A375-MR1) and with MR1T cells. On day 14, mice were sacrificed and lung nodules were counted after India ink perfusion.

P<0.0001 (Unpaired Student's t-test).

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an XML file named 95083_306_1401_seq, approximately 126 KB, created Jul. 16, 2023, and is incorporated by reference herein in its entirety.
SEQ ID NO 001: amino acid TCR alpha chain clone 1 (MCA2E7)
SEQ ID NO 002: amino acid TCR beta chain clone 1 (MCA2E7)
SEQ ID NO 003: amino acid TCR alpha chain clone 2 (MCA3C3)
SEQ ID NO 004: amino acid TCR beta chain clone 2 (MCA3C3)
SEQ ID NO 005: amino acid TCR alpha chain clone 3 (CHO9A4)
SEQ ID NO 006: amino acid TCR beta chain clone 3 (CHO9A4)
SEQ ID NO 007: nucleic acid T cell receptor
SEQ ID NO 008: nucleic acid T cell receptor
SEQ ID NO 009: nucleic acid T cell receptor
SEQ ID NO 010: nucleic acid T cell receptor
SEQ ID NO 011: nucleic acid T cell receptor
SEQ ID NO 012: nucleic acid T cell receptor
SEQ ID NO 013: amino acid TCR alpha chain clone 4 (DGA4)
SEQ ID NO 014: amino acid TCR alpha chain clone 5 (DGA28)
SEQ ID NO 015: amino acid TCR alpha chain clone 6 (DGB70)
SEQ ID NO 016: amino acid TCR alpha chain clone 7 (DGB129)
SEQ ID NO 17: amino acid TCR alpha chain clone 8 (CH9A3)
SEQ ID NO 018: amino acid TCR alpha chain clone 9 (JM64-8)
SEQ ID NO 019: amino acid TCR alpha chain clone 10 (JMA)
SEQ ID NO 020: amino acid TCR alpha chain clone 11 (LMB1F3)
SEQ ID NO 021: amino acid TCR alpha chain clone 12 (MCA3D9)
SEQ ID NO 022: amino acid TCR alpha chain clone 13 (TC5A87)
SEQ ID NO 023: amino acid TCR alpha chain clone 14 (SMC3)
SEQ ID NO 024: amino acid TCR alpha chain clone 15 (TRA44)
SEQ ID NO 025: amino acid TCR beta chain clone 4 (DGA4)
SEQ ID NO 026: amino acid TCR beta chain clone 5 (DGA28)
SEQ ID NO 027: amino acid TCR beta chain clone 6 (DGB70)
SEQ ID NO 028: amino acid TCR beta chain clone 7 (DGB129)
SEQ ID NO 029: amino acid TCR beta chain clone 8 (CH9A3)
SEQ ID NO 030: amino acid TCR beta chain clone 9 (JM64-8)
SEQ ID NO 031: amino acid TCR beta chain clone 10 (JMA)
SEQ ID NO 032: amino acid TCR beta chain clone 11 (LMB1F3)
SEQ ID NO 033: amino acid TCR beta chain clone 12 (MCA3D9)
SEQ ID NO 034: amino acid TCR beta chain clone 13 (TC5A87)
SEQ ID NO 035: amino acid TCR beta chain clone 14 (SMC3)
SEQ ID NO 036: amino acid TCR beta chain clone 15 (TRA44)
SEQ ID NO 037: nucleic acid T cell receptor
SEQ ID NO 038: nucleic acid T cell receptor
SEQ ID NO 039: nucleic acid T cell receptor
SEQ ID NO 040: nucleic acid T cell receptor
SEQ ID NO 041: nucleic acid T cell receptor
SEQ ID NO 042: nucleic acid T cell receptor
SEQ ID NO 043: nucleic acid T cell receptor
SEQ ID NO 044: nucleic acid T cell receptor
SEQ ID NO 045: nucleic acid T cell receptor
SEQ ID NO 046: nucleic acid T cell receptor
SEQ ID NO 047: nucleic acid T cell receptor
SEQ ID NO 048: nucleic acid T cell receptor
SEQ ID NO 049: nucleic acid T cell receptor
SEQ ID NO 050: nucleic acid T cell receptor
SEQ ID NO 051: nucleic acid T cell receptor
SEQ ID NO 052: nucleic acid T cell receptor
SEQ ID NO 053: nucleic acid T cell receptor
SEQ ID NO 054: nucleic acid T cell receptor
SEQ ID NO 055: nucleic acid T cell receptor
SEQ ID NO 056: nucleic acid T cell receptor
SEQ ID NO 057: nucleic acid T cell receptor
SEQ ID NO 058: nucleic acid T cell receptor
SEQ ID NO 059: nucleic acid T cell receptor
SEQ ID NO 060: nucleic acid T cell receptor
SEQ ID NO 061: amino acid TCR gamma chain clone (1) (MGDA1G5)
SEQ ID NO 062: amino acid TCR delta chain clone (1) (MGDA1G5)
SEQ ID NO 063: nucleic acid T cell receptor
SEQ ID NO 064: nucleic acid T cell receptor

### DETAILED DESCRIPTION OF THE INVENTION

### Definitions

The term MR1 in the context of the present specification refers to either the MR1 gene (Entrez 3140) or the MR1 gene product (Uniprot Q95460).

MR1 in the physiological context of a non-tumour bearing patient presents bacterial riboflavin by-products (above referred to as “exogenous microbial-derived antigens”) and presents them to mucosal invariant T cells.

An MR1-expressing cancer cell presents a particular cancer antigen, or a number of particular cancer antigens, on MR1.

The term MR1T cell in the context of the present specification refers to a T cell that expresses a T cell receptor capable of binding specifically to an MR1 molecule presented by a cancer cell.

The term MR1T cell receptor in the context of the present specification refers to a T cell receptor capable of binding specifically to an antigen presented by a cancer cell in association with an MR1 molecule.

A TCR sequence or TCR molecule described herein comprises, to be fully functional, a TCR alpha and a TCR beta polypeptide chain, or a TCR gamma and a TCR delta polypeptide chain. If reference is made to a TCR alpha or beta polypeptide having a particular sequence, it is understood that in order for this to be fully functional in the methods and cells described herein, it requires the presence of a complementary (beta or alpha, respectively) polypeptide chain. The same applies, mutatis mutandis, to the gamma delta pairing. Mention of a specific TCR alpha, beta, gamma or delta sequence implies the possibility that it is paired with the TCR sequence with which it is paired in the original clone as described herein, or a sequence of certain identity to the original pairing sequence, as specified herein. Mention of a specific TCR alpha, beta, gamma or delta sequence also implies the possibility that it is paired with another pairing TCR sequence.

The recognition of MR1-presented cancer antigens is effected mainly through CDR3 sequences. Wherein a TCR sequence characterized only by a specific CDR3 sequence is mentioned herein, it is implied that the TCR sequence is a full alpha, beta, gamma or delta TCR sequence as provided herein, and a resulting TCR molecule is paired with an appropriate second sequence.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively. Reference to identical sequences without specification of a percentage value implies 100% identical sequences (i.e. the same sequence).

In the present specification, the term positive, when used in the context of expression of a marker, refers to expression of an antigen assayed by a fluorescent labelled antibody, wherein the fluorescence is at least 30% higher (≥30%), particularly ≥50% or ≥80%, in median fluorescence intensity in comparison to staining with an isotype-matched antibody which does not specifically bind the same target. Such expression of a marker is indicated by a superscript “plus” (1, following the name of the marker, e.g. CD4⁺.

In the present specification, the term negative, when used in the context of expression of a marker, refers to expression of an antigen assayed by a fluorescent labelled antibody, wherein the median fluorescence intensity is less than 30% higher, particularly less than 15% higher, than the median fluorescence intensity of an isotype-matched antibody which does not specifically bind the same target. Such expression of a marker is indicated by a superscript minus (−), following the name of the marker, e.g. CD127⁻.

The term nucleic acid expression vector in the context of the present specification relates to a plasmid or a viral genome, which is used to transfect (in case of a plasmid) or transduce (in case of a viral genome) a target cell with a certain gene of interest. The gene of interest is under control of a promoter sequence and the promoter sequence is operational inside the target cell, thus, the gene of interest is transcribed either constitutively or in response to a stimulus or dependent on the cell's status. In certain embodiments, the viral genome is packaged into a capsid to become a viral vector, which is able to transduce the target cell.

A first aspect of the invention relates to a method of identifying and/or isolating a T cell that expresses a T cell receptor capable of binding specifically to a cancer antigen presented by a cancer cell in association with a non-polymorphic MHC I-related MR1 antigen-presenting molecule. This method comprises the steps of
a. providing a preparation of T cells isolated from a patient or a healthy donor, then
b. contacting, particularly co-culturing, this preparation of isolated T cells with cancer cells expressing MR1 protein in the absence of exogenous microbial-derived antigens in a contacting step, then
c. isolating a T cell that is specifically reactive to said cancer cells in an MR1-dependent manner in an isolation step.

In certain embodiments, the contacting step comprises an expansion step, wherein the preparation of isolated T cells is expanded in the presence of cancer cells expressing MR1. In certain particular embodiments, the cancer cells are irradiated in order to prohibit their growth prior to being brought into contact with the T cells. This is going to be advantageous if the two cell types are meant to be kept in co-culture for extended periods of time and overgrowing of the culture by the cancer cells is to be avoided.

In other settings, particularly in clinical use (see below), where culturing times are short, the cancer cells may be used without irradiation.

In certain embodiments, the expansion step is conducted in the presence of IL-2, IL-7 and IL-15.

In certain embodiments, the isolation step comprises staining with one or more ligands, in particular one or more (monoclonal) antibodies specific for a cell surface marker selected from CD3, CD69, CD137, CD150, and/or ICOS). In particularly preferred embodiments the isolation step comprises selecting CD3⁺CD137⁺, and/or CD3⁺CD69⁺, and/or CD3⁺CD150⁺, and/or CD3⁺ ICOS⁺ T cells, followed by flow cytometric analysis and cell sorting, particularly by using FACS or magnetic separation (MACS). Here positive expression (+) of a marker means at least 30% increase of the median fluorescence intensity over staining with isotype-matched antibody which does not specifically bind the same cell. In other words, T cells that express CD3 and CD137, and/or CD3 and CD69, and/or CD3 and CD150, and/or CD3 and ICOS are isolated using FACS or MACS. The skilled person is aware that in instances where the cells are isolated via FACS, cells that are positive for the expression of two (or more) different markers can be isolated in a single step. If magnetic separation is used, two separate steps have to be performed to isolate cells that are positive for the expression of two different markers.

The isolating step comprises selecting T cells that display MR1-restricted activity. In other words, this step comprises isolating T cells that are activated by an antigen presented on MR1.

In certain embodiments, the isolating step comprises selecting T cells that exhibit 2× increased expression of a cytokine selected from IFN-γ and/or GM-CSF release when stimulated with cells expressing MR1 compared to stimulation with cells not expressing MR1.

The skilled person is aware that an MR1-expressing cancer cell presents a particular cancer antigen, or a number of particular cancer antigens, on MR1.

In certain embodiments, the isolating step comprises selecting T cells that exhibit 2× increased expression of a cytokine selected from IFN-γ and/or GM-CSF release when stimulated with tumour cells expressing MR1 compared to stimulation with tumour cells (of the same origin, or same cell line) not expressing MR1.

Reactive cells are those that, in response to being contacted by an MR1-expressing cancer cell (presenting a cancer antigen in an MR1-restricted fashion), upregulate activation markers (particularly the markers cited in the preceding paragraphs), release cytokines and start to proliferate.

In other words, T cells that display MR1-restricted activity are T cells that can be activated by a tumour-associated antigen displayed by MR1.

These cells can be sorted by fluorescence activated cell sorting (FACS) after staining with the appropriate fluorescently labelled antibodies specific for the marker, or by sorting by magnetic beads labelled with the appropriate antibodies (which is the usual sorting method in a clinical setting).

In certain embodiments, the isolating step comprises expanding individual clones of the cells sorted as a function of their activation status, and then selecting T cell clones that display MR1-restricted activity, particularly cells that exhibit 2× increased expression of a cytokine selected from IFN-γ and/or GM-CSF when stimulated with cells expressing MR1 compared to stimulation with cells not expressing MR1.

In certain embodiments, the method further includes determining a nucleic acid sequence encoding a T cell receptor of the T cell isolated in the isolation step. In certain embodiments, the method includes determining two nucleic acid sequences encoding both T cell receptor chains of the T cell isolated in the isolation step.

Another aspect of the invention relates to a method of producing a preparation of transgenic MR1T cells reactive to MR1 in the absence of exogenous antigens. The method encompasses firstly, determining which T cell receptors are most likely to be reactive to a particular MR1-expressing cancer in a patient, then preparing a T cell population expressing these specific T cell receptor genes from expression constructs transferred into the cells, and administering these engineered T cells into the patient.

This method comprises the steps of
a. providing a tumour sample obtained from a patient;
b. contacting said tumour sample with a plurality of MR1T cell receptor molecule reactive to MR1, either
   presented on a plurality of T cell clones, wherein each T cell clone is characterized by an MR1T cell receptor molecule reactive to MR1; or
   as soluble MR1T cell receptor molecules that are labelled, and their recognition is assayed in a non-cell-dependent fashion;
c. identifying a number of T cell clone(s) specifically reactive to said tumour sample;
d. providing a T cell preparation, particularly a T cell preparation obtained from the same patient;
e. introducing a nucleic acid expression construct encoding an MR1-reactive T cell receptor molecule expressed on a T cell clone identified as being specifically reactive to said tumour sample in step c into said T cell preparation, yielding a transgene T cell preparation.

The transgene T cell preparation to the patient could thus be administered to the patient.

Another aspect of the invention relates to a TCR heterodimer derived from one of the MR1T cells reactive to tumor derived antigens described herein. Such TCR heterodimers are of use of transgenic receptors conferring specificity to tumour antigens to a recipient cell to provide an MR1-specific T cell clone, or may be produced in a soluble form, and optionally multimerized, for example in a tetramer format. In some embodiments, the TCR heterodimer is expressed by means of recombinant gene technology to provide a recombinant cell, or a population of cells expressing a TCRα chain in complex with a TCRβ chain on the cell surface. In particular embodiments, the recombinant cell is a T cell.

In particular embodiments of the claimed invention, the TCR heterodimer is made of either a paired TCR alpha (TCRα) chain (polypeptide) and TCR beta (TCRβ) chain, or a paired TCR gamma (TCRγ) chain and TCR delta (TCRδ) chain. TCR chains are formed by recombination of multiple genes to form a polypeptide comprising both a variable region, important for antigen recognition, and a constant region contributing to functional protein signaling. The extracellular portion of the TCR comprises hypervariable complementarity determining regions (CDR) surrounded by a less variable framework domains; a first CDR (CDR1) region, a second framework region, a second CDR (CDR2) region, a third framework region, and a third CDR (CDR3) region. The pair of complementary hypervariable CDR3 regions in a TCR heterodimer are a unique identifier of an individual T cell clone, conferring specificity to a specific antigenic molecule presented in the context of an MHC protein on the surface of an antigen presenting cell. The constant region of the TCR comprises a connecting peptide, a transmembrane domain, and an intracellular domain.

In additional embodiments of the claimed invention, the variable domain sequence of the full TCR sequences provided herein is used to convey specificity to a TCR heterodimer or polypeptide as defined herein. The TCR amino acid sequences provided herein comprise at the N-terminal end a leader signal (cleaved after synthesis), followed by a variable domain (V) and a constant domain (C). These domains have a conserved structure, which permit numbering of the amino acid residues, and comparisons of TCR chain amino acid sequences, within and among sequences, according to standard numbering sequences such as the Kabat, or IMGT systems. Identification and numbering of TCR chain domain sequences may be carried out for example, by entering the full amino acid, (or nucleic acid sequence encoding said amino acid sequence) into an online tool such as that provided by the international ImMunoGeneTics (IMGT) information system. Alternatively, CDR, or variable domain sequences may be annotated within a full TCR chain sequence using software packages such as MiTCR, MiXCR, or Immcantation—Change-O (Bolotin et al. 2013, Nat. Meth. 10:813; Bolotin et al. 2015, Nat. Meth. 12:380; Gupta et al. 2015, Bioinformatics 31:3356). Such tools and software packages known in the art can annotate the variable domain encoding the first framework region, the CDR1, the second framework region, the CDR2, the third framework region, and the junction comprising the CDR3 which indicates the C-terminal end of the variable domain.

In certain embodiments, each of the TCR heterodimers, recombinant cells, MR1-specific T cell clones or isolated, soluble monomeric or labelled and multimerized soluble T cell receptors is characterized by a TCRα polypeptide and said TCRβ polypeptide having a TCRα CDR3 and a TCRβ CDR3 selected from SEQ ID NO 065 and SEQ ID NO 080, or SEQ ID NO 066 and SEQ ID NO 081, or SEQ ID NO 067 and SEQ ID NO 082, or SEQ ID NO 068 and SEQ ID NO 083, or SEQ ID NO 069 and SEQ ID NO 084, or SEQ ID NO 070 and 85, or SEQ ID NO 071 and SEQ ID NO 086, or SEQ ID NO 072 and SEQ ID NO 087, or SEQ ID NO 073 and SEQ ID NO 088, or SEQ ID NO 074 and SEQ ID NO 089, or SEQ ID NO 075 and SEQ ID NO 090, or SEQ ID NO 076 and SEQ ID NO 091, or SEQ ID NO 077 and SEQ ID NO 092, or SEQ ID NO 078 and SEQ ID NO 093, or SEQ ID NO 079 and SEQ ID NO 094.

The basis of the CDR3 assignation that forms the basis of SEQ ID NO 65 to 94 as used in the preceding paragraph includes the IMGT type defined CDR3 residues and certain conserved adjacent residues. The skilled person knows to derive other CDR3 sequences from the TCR sequences provided herein, such as the underlined and bold face sequences of the section designated “Sequences” below.

In certain embodiments, each of the TCR heterodimers, recombinant cells, MR1-specific T cell clones or isolated, soluble monomeric or labelled and multimerized soluble T cell receptors is characterized by a CDR3 sequence tract selected from any one of SEQ ID NO 065 to SEQ ID NO 096.Likewise, the CDR3 sequence tract can be characterized by a sequence identical to a sequence selected from any one of SEQ ID NO 065 to SEQ ID NO 096 with one or two amino acid substitutions. In particular embodiments, the substitutions made to the CDR3 sequence are selected according to the following substitution rules:
glycine (G) and alanine (A) are interchangeable; valine (V), leucine (L), and isoleucine (I) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) are interchangeable.

In certain embodiments, each of the TCR heterodimers, recombinant cells, MR1-specific T cell clones or isolated, labelled and multimerized soluble T cell receptors is characterized by a CDR3 sequence tract selected from any one of SEQ ID NO 065 to SEQ ID NO 096 wherein the sequence comprises a maximum total of 0,1, or 2 substitutions in the three N and/or C terminal positions according to the above substitution rules in the three N and/or C terminal positions, whereas the central amino acids of the CDR3 sequence as indicated are not changed It is known that the central part of the CDR3 sequence contributes most to antigen binding or recognition specificity.

In certain embodiments, each of the TCR heterodimers, recombinant cells, MR1-specific T cell clones or isolated, labelled and multimerized soluble T cell receptors are characterized by a TCRα variable domain identical to the TCRβ variable domains comprised within the sequences SEQ ID NO 001 and SEQ ID NO 002; or, SEQ ID NO 003 and SEQ ID NO 004; or SEQ ID NO 005 and SEQ ID NO 006, SEQ ID NO 013 and SEQ ID NO 025; or SEQ ID NO 014 and SEQ ID NO 026; or SEQ ID NO 015 and SEQ ID NO 027; or SEQ ID NO 016 and SEQ ID NO 028; or SEQ ID NO 017 and SEQ ID NO 029; or SEQ ID NO 018 and SEQ ID NO 030; or SEQ ID NO 019 and SEQ ID NO 031; or SEQ ID NO 20 and SEQ ID NO 032; or SEQ ID NO 021 and SEQ ID NO 033; or SEQ ID NO 022 and SEQ ID NO 034; or SEQ ID NO 023 and SEQ ID NO 035; or SEQ ID NO 024 and SEQ ID NO 036, as annotated in the Sequences section below. In other embodiments, each of the TCR heterodimers, MR1-specific T cell clones or isolated, labelled and multimerized soluble T cell receptors are characterized by a TCRγ variable domain identical to the TORO variable domains comprised within the sequences SEQ ID NO 061 and SEQ ID NO 062.

In certain embodiments, each of the TCR heterodimers, recombinant cells, MR1-specific T cell clones or isolated, labelled and multimerized soluble T cell receptors is characterized by, or encoded by a nucleic acid sequence selected from SEQ ID NO 007 to SEQ ID NO 012 or SEQ ID NO 037 to SEQ ID NO 060 or SEQ ID NO 063 to SEQ ID NO 064 and/or an amino acid sequence selected from SEQ ID NO 001 to SEQ ID 006 or SEQ ID NO 013 to SEQ ID NO 036 or SEQ ID NO 061 to SEQ ID NO 062.

In certain embodiments, each of the TCR heterodimers, recombinant cells, MR1-specific T cell clones or isolated, labelled and multimerized soluble T cell receptors is characterized by “The same biological activity” in this context refers to the ability of a recombinant TCR sequence to recognize (or contribute in the recognition of) an MR1 molecule presenting a cancer antigen on a cancer cell. Assays and methods to determine such interaction are described herein.

In certain embodiments, each of the TCR heterodimers, recombinant cells, MR1-specific T cell clones or isolated, labelled and multimerized soluble T cell receptors is characterized by a T cell receptor α chain nucleic acid sequence selected from SEQ ID NO 007, 009 to 011 or SEQ ID NO 037 to SEQ ID NO 048 and/or an amino acid sequence selected from SEQ ID NO 001, 003 to 005 or SEQ ID NO 013 to SEQ ID NO 024.

In certain embodiments, each of the TCR heterodimers, recombinant cells, MR1-specific T cell clones or isolated, soluble monomeric or labelled and multimerized soluble T cell receptors is characterized by a T cell receptor a chain amino acid sequence at least 85% (≥90%, 95%, 98) identical to SEQ ID NO 001, 003 to 005 or SEQ ID NO 013 to SEQ ID NO 024 and having the same biological activity, particularly an amino acid sequence at least 85% (≥90%, 95%, 98) identical to SEQ ID NO 001, 003 to 005 or SEQ ID NO 013 to SEQ ID NO 024 comprising a CDR sequence selected from SEQ ID NO 065 to SEQ ID NO 079

In certain embodiments, each of the TCR heterodimers, recombinant cells, MR1-specific T cell clones or isolated, labelled and multimerized soluble T cell receptors is characterized by a T cell receptor β chain nucleic acid sequence selected from SEQ ID NO 008, 010 to 012 or SEQ ID NO 049 to SEQ ID NO 060 and/or an amino acid sequence selected from SEQ ID NO 002, 004 to 006 or SEQ ID NO 025 to SEQ ID NO 036.

In certain embodiments, each of the TCR heterodimers, recombinant cells, MR1-specific T cell clones or isolated, labelled and multimerized soluble T cell receptors is characterized by a T cell receptor β chain amino acid sequence at least 85% (≥90%, 95%, 98) identical to SEQ ID SEQ ID NO 002, 004 to 006 or SEQ ID NO 025 to SEQ ID NO 036 and having the same biological activity, particularly an amino acid sequence at least 85% (≥90%, 95%, 98) identical to SEQ ID NO 002, 004 to 006 or SEQ ID NO 025 to SEQ ID NO 036 comprising a CDR sequence selected from SEQ ID NO 080 to SEQ ID NO 094.

In certain embodiments, each of the TCR heterodimers, recombinant cells, MR1-specific T cell clones or isolated, labelled and multimerized soluble T cell receptors is characterized by a T cell receptor γ chain nucleic acid sequence SEQ ID NO 61 and/or an amino acid sequence SEQ ID NO 063, or a sequence at least 85% (≥90%, 95%, 98) identical thereto and having the same biological activity, particularly an amino acid sequence at least 85% (≥90%, 95%, 98) identical to SEQ ID NO 063 and comprising a CDR3 of SEQ ID NO 095

In certain embodiments, each of the TCR heterodimers, recombinant cells, MR1-specific T cell clones or isolated, labelled and multimerized soluble T cell receptors is characterized by a T cell receptor δ chain nucleic acid sequence SEQ ID NO 64 and/or an amino acid sequence SEQ ID NO 062, or an amino acid sequence at least 85% (≥90%, 95%, 98) identical to SEQ ID NO 062 and comprising a CDR3 of SEQ ID NO 096.

In certain embodiments, each of the TCR heterodimers, recombinant cells, MR1-specific T cell clones or isolated, labelled and multimerized soluble T cell receptors is characterized by a T cell receptor α chain and β chain nucleic acid sequence pair selected from the pairs: SEQ ID NO 007 and SEQ ID NO 008, SEQ ID NO 009 and SEQ ID NO 010, SEQ ID NO 011 and SEQ ID NO 012, SEQ ID NO 037 and SEQ ID NO 049, SEQ ID NO 038 and SEQ ID NO 050, SEQ ID NO 039 and SEQ ID NO 051, SEQ ID NO 040 and SEQ ID NO 052, SEQ ID NO 041 and SEQ ID NO 053, SEQ ID NO 042 and SEQ ID NO 054, SEQ ID NO 043 and SEQ ID NO 055, SEQ ID NO 044 and SEQ ID NO 056, SEQ ID NO 045 and SEQ ID NO 057, SEQ ID NO 046 and SEQ ID NO 058, SEQ ID NO 047 and SEQ ID NO 059 or SEQ ID NO 048 and SEQ ID NO 060.

In certain embodiments, each of the TCR heterodimers, recombinant cells, MR1-specific T cell clones or isolated, labelled and multimerized soluble T cell receptors is characterized by a T cell receptor γ chain and δ chain nucleic acid sequence pair selected from SEQ ID NO 063 and SEQ ID NO 064, or a sequence at least 85% (≥90%, 95%, 98) identical thereto having the same biological activity as the unmutated pair.

In certain embodiments, each of the TCR heterodimers, recombinant cells, MR1-specific T cell clones or isolated, labelled and multimerized soluble T cell receptors is characterized by a T cell receptor α chain and β chain amino acid sequence pair selected from the pairs: SEQ ID NO 001 and SEQ ID NO 002, SEQ ID NO 003 and SEQ ID NO 004, SEQ ID NO 005 and SEQ ID NO 006, SEQ ID NO 013 and SEQ ID NO 025, SEQ ID NO 014 and SEQ ID NO 026, SEQ ID NO 015 and SEQ ID NO 027, SEQ ID NO 016 and SEQ ID NO 028, SEQ ID NO 017 and SEQ ID NO 029, SEQ ID NO 018 and SEQ ID NO 030, SEQ ID NO 019 and SEQ ID NO 031, SEQ ID NO 20 and SEQ ID NO 032, SEQ ID NO 021 and SEQ ID NO 033, SEQ ID NO 022 and SEQ ID NO 034, SEQ ID NO 023 and SEQ ID NO 035, SEQ ID NO 024 and SEQ ID NO 036, or a pair selected from the pairs given in the previous paragraph, wherein each of the partners may have a sequence at least 85% (≥90%, 95%, 98) identical to the indicated SEQ ID NO and the pair has the same biological activity as the unmutated pair.

In particular embodiments, each of the amino acid sequences comprises a CDR3 sequence identical to the indicated SEQ ID NO as can be inferred from the table below.

In certain embodiments, each of the TCR heterodimers, recombinant cells, MR1-specific T cell clones or isolated, labelled and multimerized soluble T cell receptors is characterized by a T cell receptor γ chain and δ chain amino acid sequence pair selected from SEQ ID NO 061 and SEQ ID NO 062.

In certain embodiments, the T cell preparation according to the invention is obtained from the same patient (autologous adoptive T cell therapy). This method has the advantage of avoiding the risk of adverse reactions, particularly an allo-immune reaction driven by the endogenous T cell receptors of the engineered T cell preparation.

In certain embodiments, the T cell preparation according to the invention is obtained from another subject, particularly a HLA-matched subject (allogeneic adoptive T cell therapy). While depending on the quality of the HLA match, the risk of alloimmunity may be significant, the logistics and procedural advantages of having a large selection of pre-made TC preparations to select from may facilitate this therapy to a vastly larger patient community in comparison to the far higher costs and regulatory hurdles of a bespoke, patient-individual therapy.

Introduction of the MR1T cell receptor expression construct into the T cell preparation may be achieved by lentiviral transduction, which the inventors have routinely used in their work on MR1T cells, or by standard methods of DNA expression vector (plasmid) or RNA transfection. The skilled person is aware of the relevant protocols and procedures.

Optionally, the transgene T cell preparation may be kept in culture for some time prior to being administered to the patient in order to expand their number and, again optionally, to further stimulate their differentiation into a particularly desired T cell subset.

In certain embodiments, the T cell preparation obtained from said patient is obtained from peripheral blood of the patient, particularly wherein said T cell preparation is obtained by selecting peripheral blood mononuclear cells (PBMC) for expression of one or several T cell markers selected from the group containing CD4, CD8, CD27, CD45RA and CD57.

In certain embodiments, the T cell preparation obtained from said patient is obtained from a tumour biopsy followed by subsequent expansion in-vitro. In certain embodiments, T cells are expanded in the presence of phytohemagglutinin, IL-2, IL-7 and IL-15. Proliferating T cells are isolated by magnetic sorting and used for T cell receptor engineering or for cloning and isolation of tumour-specific MR1-restricted T cells. The isolated MR1T cells are used for TCR gene cloning.

The plurality of MR1-specific T cell clones can be prepared in advance of the procedure and held in form of a library or panel for ad-hoc use whenever the need for rapid characterization of a tumour arises. This step is essentially an identification of the MR1-specific T cell receptor molecules that will recognize a particular tumour entity.

Alternatively, soluble MR1T TCRs may be generated and multimerized (see Subbramanian et al. Nature Biotechnology, 22, 1429, (2004)). TCR multimers will be labeled with fluorochromes and used to stain tumour cells isolated from tumour biopsies. Binding of soluble MR1T TCR multimers will indicate the capacity of that MR1T TCR to recognize tumour cells and thus will facilitate selection of the MR1T TCRs suitable for gene therapy in that patient.

Another aspect of the invention relates to an expression vector comprising, and leading to the transcription of, a nucleic acid sequence encoding a functional T cell receptor heterodimer, or a T cell receptor α chain capable of forming a functional T cell receptor heterodimer together with a T cell receptor β chain, and/or a T cell receptor β chain capable of forming a functional T cell receptor heterodimer together with a T cell receptor α chain. Of note, also MR1-specific γ-δ heterodimers have been found by the inventors, so the same applies to these chains.

In embodiments where the expression vector comprises a nucleic acid sequence encoding a T cell receptor α chain or a T cell receptor β chain (or a γ or δ chain), two different expression vectors (one encoding an α chain (γ chain) and one encoding a β chain (δ chain)) have to be introduced into a cell in order to enable expression of a functional T cell receptor heterodimer by said cell. The T cell receptor heterodimer specifically binds to an MR1 molecule, wherein said MR1 molecule is expressed on a tumour cell and presents a tumour-associated antigen.

The expression of the above mentioned nucleic acid sequences is controlled by a promoter sequence operable in a mammalian cell, particularly a human T-cell. In certain embodiments, the promoter is a constitutively activated promoter, for example the CMV immediate early promoter commonly used in molecular biology. In certain other embodiments, the promoter is an inducible promoter.

In certain embodiments of this aspect of the invention, the nucleic acid sequence comprised in the expression vector is or comprises a nucleic acid sequence that is selected from SEQ ID NO 007, SEQ ID NO 009 or SEQ ID NO 011, and/or encodes an amino acid sequence selected from SEQ ID NO 001, SEQ ID NO 003 or SEQ ID NO 005 (alpha chains).

In certain embodiments of this aspect of the invention, the nucleic acid sequence comprised in the expression vector is or comprises a nucleic acid sequence that is selected from SEQ ID NO 008, SEQ ID NO 010 or SEQ ID NO 012 and/or encodes an amino acid sequence selected from SEQ ID NO 002, SEQ ID NO 004 or SEQ ID NO 006 (beta chains).

Another aspect of the invention relates to a nucleic acid sequence encoding a functional T cell receptor heterodimer. The T cell receptor heterodimer specifically binds to a non-polymorphic MHC I-related (MR1) antigen-presenting molecule expressed on a tumour cell presenting a tumour-associated antigen.

In certain embodiments, the nucleic acid sequence encodes a T cell receptor α chain and is selected from SEQ ID NOs SEQ ID NO 007, SEQ ID NO 009 or SEQ ID NO 011, or encodes a T cell receptor α chain specified by an amino acid sequence selected from SEQ ID NO 001, SEQ ID NO 003 or SEQ NO ID 005.

In certain embodiments, the nucleic acid sequence encodes a T cell receptor β chain and is selected from SEQ ID NO 008, SEQ ID NO 010 or SEQ ID NO 012 or encodes a T cell receptor β chain specified by an amino acid sequence selected from SEQ ID NO 002, SEQ ID NO 004 or SEQ ID NO 006, or a sequence at least 85% (≥90%, 95%, 98) identical to an amino acid sequence selected from SEQ ID NOs 001 to 006 and having the same biological activity. In particular embodiments, each of the amino acid sequences comprises a CDR3 sequence selected from SEQ ID 65, 66, 67, 80, 81 and 82.

Another aspect of the invention relates to a T cell receptor protein that binds to a non-polymorphic MHC I-related MR1 antigen-presenting molecule. The MR1 molecule is expressed on a tumour cell and presents a tumour-associated antigen. In certain embodiments, the T cell receptor protein that binds to a non-polymorphic MHC I-related MR1 antigen-presenting molecule is identified by the method according to the first aspect of the invention.

In certain embodiments, the T cell receptor protein comprises a T cell receptor α chain characterized by an amino acid sequence selected from SEQ ID NO 001, SEQ ID NO 003 or SEQ NO ID 005 and a T cell receptor β chain characterized by an amino acid sequence selected from SEQ ID NO 002, SEQ ID NO 004 or SEQ ID NO 006.

Another aspect of the invention relates to a recombinant cell comprising the expression vector according to the invention, and/or the T cell receptor polypeptide according to the invention as specified in the preceding paragraphs. The skilled person is aware that in instances where the expression vector only comprises a nucleic acid sequence encoding a T cell receptor α chain (or a γ chain) or a T cell receptor β chain (or a 8 chain), but not both, two different expression vectors (one encoding an α/γ chain and one encoding a β/δ chain) have to be introduced into the recombinant cell in order to enable expression of a functional T cell receptor heterodimer by said cell. In certain embodiments, the recombinant cell is a T cell derived from peripheral blood. In certain embodiments, the recombinant cell is derived from a tumour infiltrating lymphocyte.

Yet another aspect of the invention relates to the use of the recombinant cell according to the previously specified aspect of the invention for use in a method of therapy or prevention of cancer. The method comprises administration of the recombinant cell.

In certain embodiments, the cancer is characterized by MR1 expression.

In certain embodiments, the administration is effected by adoptive T cell immunotherapy.

The invention further relates to a method of treatment, or prevention of recurrence, of cancer, comprising administration of the recombinant cell according to the invention. In certain embodiments, the cancer is characterized by MR1 expression.

In certain embodiments, the administration is achieved by adoptive T cell immunotherapy.

The invention also relates to a collection of nucleic acid sequences, wherein each member of the collection encodes a different T cell receptor α chain, T cell receptor β chain, T cell receptor γ chain, T cell receptor δ chain or a T cell receptor α chain and β chain combination, or a T cell receptor γ chain and δ chain combination, wherein said combination is capable of specifically binding to an MR1 molecule presenting a cancer antigen. The nucleic acid sequences are capable to facilitate the expression of the T cell receptor α chain, β chain, or α and β chain combination in a mammalian cell.

Such collection will be used to select transgene constructs for transfer into T cells collected from a patient. After identification of the TCR sequences that are best to fit instigate reaction to a particular set of tumour antigens presented by the tumour in the first phase of the method of treatment, the physician will need to be able to select pre-produced expression vectors from such collection manufactured under GMP, to quickly effect the gene transfer into the patient's T cells.

In certain embodiments, the collection comprises a sequence selected from SEQ 007 to SEQ ID NO 012 and/or the collection comprises sequences encoding a T cell receptor molecule (or a T cell receptor constituting α or β chain) selected from SEQ ID NO 001 to SEQ ID NO 006.

Yet another aspect of the invention relates to a collection of recombinant T cells, wherein each member of the collection expresses as a transgene a T cell receptor capable of specifically binding to an MR1 molecule presenting a cancer antigen. In certain embodiments, the collection comprises a recombinant T cell comprising a T cell receptor protein heterodimer according to the respective aspect of the invention.

The inventors identified and isolated a novel population of human MR1-restricted T cells reactive to a variety of tumour cells in MR1-dependent manner. MR1T cell clones were commonly found in the blood of different healthy individuals, expressed diverse TCR genes and did not recognize previously identified microbial or folate-derived ligands of MR1. Instead, they recognized diverse sets of yet unknown antigens isolated from tumour cells and presented by MR1. The identification and characterization of the stimulatory antigens associated with tumour cells is currently ongoing. MR1T cell clones recognized and killed different types of tumour cells, thus displaying marked anti-tumour activity in vitro. In addition, they released different combinations of Th1, Th2 and Th17 cytokines, and displayed multiple chemokine receptor expression profiles, suggesting phenotypical and functional heterogeneity. Importantly, when paired TCR α and β genes or TCR γ and δ genes isolated from individual MR1T cell clones were transferred into TCR-deficient T cells, the recipient T cells acquired the capacity to recognize MR1-expressing tumour cells, thus indicating that the MR1T cell TCR gene transfer is sufficient for this type of tumour recognition and might be used to instruct select T cells to recognize MR1-expressing tumour cells.

Taken together, these findings reveal a novel functionally diverse population of tumour-reactive human T cells restricted to non-polymorphic MR1 molecules with diverse potential role in tumour immunity, thus providing new conceptual frameworks for cancer immune surveillance and immunotherapies.

In the present specification, the following abbreviations are used: APC, antigen-presenting cell; β2m, β2 microglobulin; DC, dendritic cell; GM-CSF, granulocyte-macrophage colony-stimulating factor; HPLC, high-pressure liquid chromatography; IFN-γ, interferon-γ; mAb, monoclonal antibody; MAIT cell, mucosal associated invariant T cell; MHC, major histocompatibility complex; MR1, MHC class I-related molecule; MR1T cell, MR1-restricted T cell; PBMC, peripheral blood mononuclear cell; TCR, T cell receptor; TIL, tumour-infiltrating lymphocyte.

The invention is further illustrated by the following examples and figures, from which further embodiments and advantages can be drawn. These examples are meant to illustrate the invention but not to limit its scope.

### Examples

### Methods

Cells. The following human cell lines were obtained from American Type Culture Collection: A375 (melanoma), THP-1 (myelomonocytic leukemia), J.RT3-T3.5 (TCRβ-deficient T cell leukemia), LS 174T (colon adenocarcinoma), HCT116 (colon carcinoma), Huh7 (hepatocellular carcinoma), HEK 293 (human embryonic kidney), and CCRF-SB (acute B cell lymphoblastic leukemia). SKW-3 cells (human T cell leukemia deficient in TCRα, β, γ and δ genes) were obtained from the Leibniz-Institute DSMZ-German Collection of Microorganisms and Cell Cultures. Two representative MAIT clones (MRC25 and SMC3) and one TCR γδ clone, (G2B9) (Gober et al., The Journal of experimental medicine 197, 163-168 (2003)) were used in this study as control cells and were generated from blood of two healthy donors and maintained in culture as previously described (Lepore et al., Nat Commun 5, 3866 (2014)). MR1T cells were isolated from the peripheral blood of healthy individuals after informed consent was obtained from donors at the time of blood collection under approval of the “Ethikkommision Nordwest and Zentralschweiz/EKNZ (139/13). Briefly, T cells purified by negative selection (EasySep™ Human T Cell Enrichment Kit, StemCell) were stimulated with irradiated (80 Gray) A375-MR1 cells (ratio 2:1) once a week for three weeks. Human rIL-2 (5 U/ml; Hoffmann-La Roche), rIL-7 and rIL-15 (both at 5 ng/ml, Peprotech) were added at day +2 and +5 after each stimulation. Twelve days after the last stimulation cells were washed and co-cultured overnight with A375-MR1 cells (ratio 2:1). CD3⁺CD69⁺CD37⁺ cells were then sorted and cloned by limiting dilution in the presence of PHA (1 μg/ml, Wellcome Research Laboratories), human rIL-2 (100 U/ml, Hoffmann-La Roche) and irradiated PBMC (5×10⁵ cells/ml). In other experiments, MR1T cells clones were generated using the same protocol from sorted CD3⁺CD69⁺CD137⁺ upon a single overnight stimulation with A375-MR1 cells (ratio 2:1). T cell clones were periodically re-stimulated following the same protocol (Lepore et al., ibid.). Monocytes and B cells were purified (>90% purity) from PBMCs of healthy donors using EasySep Human CD14 and CD19 positive selection kits (Stemcell Technologies) according to the manufacturer instructions. Mo-DCs were differentiated from purified CD14+ monocytes by culture in the presence of GM-CSF and IL-4 as previously described (Lepore et al., ibid.). Human normal gut epithelial cells (GEC) were isolated from gut biopsies of tumour-free individuals according to a published protocol (Graves et al., Journal of immunological methods 414, 20-31 (2014)).

Generation of cells expressing MR1A gene covalently linked with β2m. A human MR1A cDNA construct linked to β2m via a flexible Gly-Ser linker was generated by PCR as previously described (Lepore et al., ibid.). The K43A substitution in the MR1A cDNA was introduced into the fusion construct using the following primers: MR1 K43A_f 5′-CTCGGCAGGCCGAGCCACGGGC (SEQ ID NO 097) and MR1 K43A_r 5′GCCCGTGGCTCGGCCTGCCGAG (SEQ ID NO 098). Resulting WT and mutant constructs were cloned into a bidirectional lentiviral vector (LV) (Lepore et al., ibid.). HEK 293 cells were transfected with individual LV-MR1A-82m constructs together with the lentivirus packaging plasmids pMD2.G, pMDLg/pRRE and pRSV-REV (Addgene) using Metafectene Pro (Biontex) according to manufacturer instructions. A375, and THP-1, cells were transduced by spin-infection with virus particle containing supernatant in the presence of 8 μg/ml protamine sulfate. Surface expression of MR1 was assessed by flow cytometry and positive cells were FACS sorted.

Soluble recombinant β2m-MR1-Fc fusion protein. β2m-MR1-Fc fusion construct was obtained using human MR1A-β2m construct described above as template. DNA complementary to β2m-MR1A gene was amplified by PCR using primers: β2mXhol_f 5′-CTCGAGATGTCTCGCTCCGTGGCCTTA (SEQ ID NO 099) and MR1-IgG1_r 5′-GTGTGAGTTTTGTCGCTAGCCTGGGGGACCTG (SEQ ID NO 100), thus excluding MR1 trans-membrane and intracellular domains. The DNA complementary to the hinge region and CH2-CH3 domains of human IgG1 heavy chain was generated using the following primers: Nhel-hinge-f 5′-CAGGTCCCCCAGGCTAGCGACAAAACTCACAC (SEQ ID NO 101) and IgG1Notl_r 5′-GCGGCCGCTCATTTACCCGGAGACAGGGAGA (SEQ ID NO 102) from pFUSE-hIgG1-Fc1 (InvivoGen). The β2m-MR1A and IgG1 PCR products were joined together using two-step splicing with overlap extension PCR and the resulting construct subcloned into the Xhol/Notl sites of the BCMGSNeo expression vector. CHO-K1 cells were transfected with the final construct using Metafectene Pro (Biontex), cloned by limiting dilutions and screened by ELISA for the production of β2m-MR1-Fc fusion protein. Selected clones, adapted to EX-CELL ACF CHO serum-free medium (Sigma), were used for protein production and β2m-MR1-Fc was purified using Protein-A-Sepharose (Thermo Fisher Scientific) according to manufacturer instructions. Protein integrity and purity were verified by SDS-PAGE and Western Blot using anti-MR1 mAb 25.6 (Biolegend).

Flow cytometry and antibodies. Cell surface labeling was performed using standard protocols. Intracellular labeling was performed using the True-Nuclear™ Transcription Factor Buffer Set according to the manufacturers' instructions. The following anti-human mAbs were obtained from Biolegend: CD4-APC (OKT4), CD8α-PE (TuGh4), CD161-Alexa Fluor 647 (HP-3G10), CD69-PE (FN50), CD3-PE/Cy7, Brilliant Violet-711, or Alexa-700 (UCHT1), CD137-biotin (n4b4-1), CXCR3-Brilliant Violet 421 (G025H7), CD83-biotin (HB15e), MR1-PE (26.5) and TRAV1-2-PE (10C3). CD86-FITC (2331), CCR4-PECy7 (1G1) and CCR6-PE (11A9) mAbs were from BD Pharmingen. All these mAbs were used at 5 μg/ml. Biotinylated mAbs were revealed with streptavidin-PE, -Alexa Fluor 488, or -Brilliant violet 421 (2 μg/ml, Biolegend). Samples were acquired on LSR Fortessa flow cytometer (Becton Dickinson). Cell sorting experiments were performed using an Influx instrument (Becton Dickinson). Dead cells and doublets were excluded on the basis of forward scatter area and width, side scatter, and DAPI staining. All data were analyzed using FlowJo software (TreeStar).

TCR gene analysis of MR1T cell clones. TCRα and β or gene TCRγ and δ expression by MR1T cell clones was assessed either by RT-PCR using total cDNA and specific primers, or by flow cytometry using the 10Test® Beta Mark TCR Vβ Repertoire Kit (Beckman Coulter) according to the manufacturers' instructions or panyδ TCR-specific monoclonal antibodies (B1, Biolegend). For RT-PCR, RNA was prepared using the NucleoSpin RNA II Kit (Macherey Nagel) and cDNA was synthesized using Superscript III reverse transcriptase (Invitrogen). TCRα, β, γ and δ cDNAs were amplified using sets of Vα, Vβ, Vγ and Vδ primers as directed by the manufacturer (TCR typing amplimer kit, Clontech). Functional transcripts were identified by sequencing and then analyzed using the ImMunoGeneTics information system (http://www.imgt.org).

TCR gene transfer. TCRα and β functional cDNA from the MAIT cell clone MRC25 were cloned into the Xhol/Notl sites of the BCMGSNeo expression vector (Karasuyama and Melchers Eur. J. Immunol. 1988 18:97-104) and the resulting constructs were used to co-transfect J.RT3-T3.5 cells by electroporation according to standard procedure. Transfectants expressing TRAV1-2 and CD3 were FACS sorted. The TCRα and β or TCRγ and δ functional cDNAs from MR1T clones were cloned into the Xmal/BamHI sites of a modified version of the plasmid 52962 (Addgene) expression vector. SKW-3 cells were transduced with virus particle-containing supernatant generated as described above. Cells were FACS sorted based on CD3 expression.

Fractionation of cell and whole tumour lysates. Total cell lysates were generated from a single pellet of 2.5×10⁹ THP-1 cells via disruption in water with mild sonication. The sonicated material was then centrifuged (15,000 g for 15 min at 4° C.) and the supernatant collected (S1). Next, the pellet was re-suspended in methanol, sonicated, centrifuged as before, and the supernatant obtained was pooled with the S1 supernatant. The final concentration of methanol was 10%. The total cell extract was then loaded onto a C18 Sep-Pak cartridge (Waters Corporation) and the unbound material was collected and dried (fraction E-FT). Bound material was eluted in batch with 75% (fraction E1) and 100% methanol (fraction E2). The E-FT material was re-suspended in acetonitrile/water (9:1 vol/vol) and loaded onto a NH₂ Sep-Pak cartridge (Waters Corporation). Unbound material (fraction N-FT) and 4 additional fractions were eluted with increasing quantities of water. Fraction N1 was eluted with 35% H₂O, fraction N2 with 60% H₂O, fraction N3 with 100% H₂O, and fraction N4 with 100% H₂O and 50 mM ammonium acetate (pH 7.0). All fractions were dried and then re-suspended in 20% methanol (fractions E1, E2 and N-FT) or 100% H₂O (all other fractions) prior to being stored at −70° C.

Mouse EMT6 breast tumours were prepared as described (Zippelius et al., Cancer Immunol Res 3, 236-244 (2015)). Freshly excised tumours were extensively washed in saline, weighted and 4 g masses were homogenized in 7 ml of HPLC-grade water using a Dounce tissue grinder. Tumour homogenate underwent two freeze-thaw cycles, centrifuged (3,250 g) for 10 min at 4° C., and supernatant was collected and stored at −70° C. The pellet was extracted a second time with 2 ml of HPLC-grade water, centrifuged (5,100 g) for 10 min at 4° C. and the supernatant was collected and stored at −70° C. The pellet was further extracted with 9 ml of HPLC-grade methanol for 5 min at room temperature by vortexing, centrifuged (5,100 g) for 10 min at 4° C., and supernatant collected. The three supernatants were pooled, dried, and resuspended in water:methanol (10:1). Material was fractionated using C18 and NH₂ Sep-Pak cartridges as above.

T cell activation assays. MR1-restricted T cells (5×10⁴/well unless otherwise indicated) were co-cultured with indicated target cells (5×10⁴/well) in 200 μl total volume in duplicates or triplicates. T cells were cultured with indicated APCs for 24 h. In some experiments, anti-MR1 mAbs (clone 26.5) or mouse IgG2a isotype control mAbs (both at 30 μg/ml) were added and incubated for 30 min prior to the addition of T cells. E. coli lysate was prepared from the DH5a strain (Invitrogen) grown in LB medium and collected during exponential growth. Bacterial cells were washed twice in PBS and then lysed by sonication. After centrifugation (15,000 g for 15 min), the supernatant was collected, dried, and stored at −70° C. APCs were pulsed for 4 h with E. coli lysate equivalent to 10⁸ CFU/ml (unless otherwise indicated) before addition of T cells. In some experiments, APCs were pre-incubated with 6-FP or Ac-6-FP (Schircks Laboratories) for 4 h before co-culture with T cells. In control experiments with TCR γδ cells expressing TCR Vγ9 and Vδ2 chains, the APCs were first treated for 6 h with zoledronate (10 μg/ml) prior to T cell addition. Activation experiments with plate-bound recombinant human β2m-MR1-Fc were performed by coating β2m-MR1-Fc onto 96 well plates (4 μg/ml) and loading with cartridge-purified cell lysates for 4 h at 37° C. before washing twice and adding T cells. Supernatants were collected after 24 h and IFN-γ or GM-CSF were assessed by ELISA. Multiple cytokines and chemokines in cell culture supernatants were analyzed using the Milliplex MAP human cytokine/chemokine magnetic bead panel—Premixed 41 plex (HCYTMAG-60K-PX41; Merck Millipore) according to the manufacturer's instructions. Samples were acquired on a Flexmap 3D system (Merck Millipore) and Milliplex analyst software was used to determine mean fluorescence intensity and analyte concentration.

Killing of tumour cells. Killing assays were performed using target cell lines (2×10⁴ cells/rill) incubated either alone or with T cells at different E/T ratios for 24 h, in the presence or absence of anti-MR1 mAb (30 μg/ml, clone 26.5). The target cells were stained with PE— Annexin V (BD) and propidium iodide (PI) (Sigma-Aldrich), as previously described (2). T cells were identified by staining with anti-CD3 mAbs and excluded from the analysis. Apoptosis was evaluated as follows: Annexin V⁺PI⁺=advanced apoptosis and Annexin V⁻PI⁺=necrosis. The percentage of apoptotic+necrotic cells in the absence of T cells (spontaneous apoptosis; no T cells) is also shown.

Statistics. Data were analyzed using Unpaired Student's t-test (Prism 6, GraphPad software).

### Identification and Characterization of Novel Tumour-Reactive MR1-Restricted T Cells in Healthy Donors

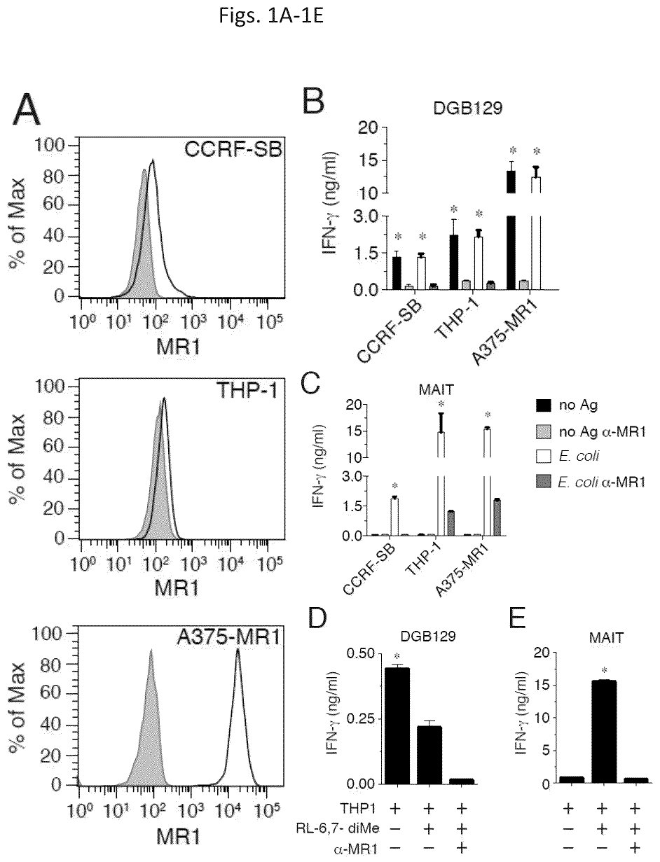

The inventors detected an atypical MR1-restricted T cell clone that did not react to microbial ligands during earlier studies on the repertoire of human MAIT cells. This T cell clone (DGB129) recognized cell lines constitutively displaying surface MR1 (CCRF-SB lymphoblastic leukemia cells, or THP-1 monocytic leukemia cells; FIG. 1A) or transfected with the MR1 gene (A375 melanoma cells; A375-MR1; FIG. 1A) in the absence of any exogenously added antigens (FIG. 1B). Sterile recognition of MR1+ target cells was fully inhibited by blocking with anti-MR1 monoclonal antibodies (mAbs) (FIG. 1B), and thus resembled the MAIT cell response to E. coli-derived antigens assessed in parallel (FIG. 1C). Importantly, DGB129 T cells also failed to recognize the synthetic MAIT cell agonist 6,7-dimethyl-8-D-ribityllumazine (RL-6,7-diMe; FIG. 1D), differently from a control MAIT cell clone, which instead was stimulated in MR1-dependent manner by this compound (FIG. 1E). DGB129 cells did not express the canonical semi-invariant TCR typical of MAIT cells (Table 1).

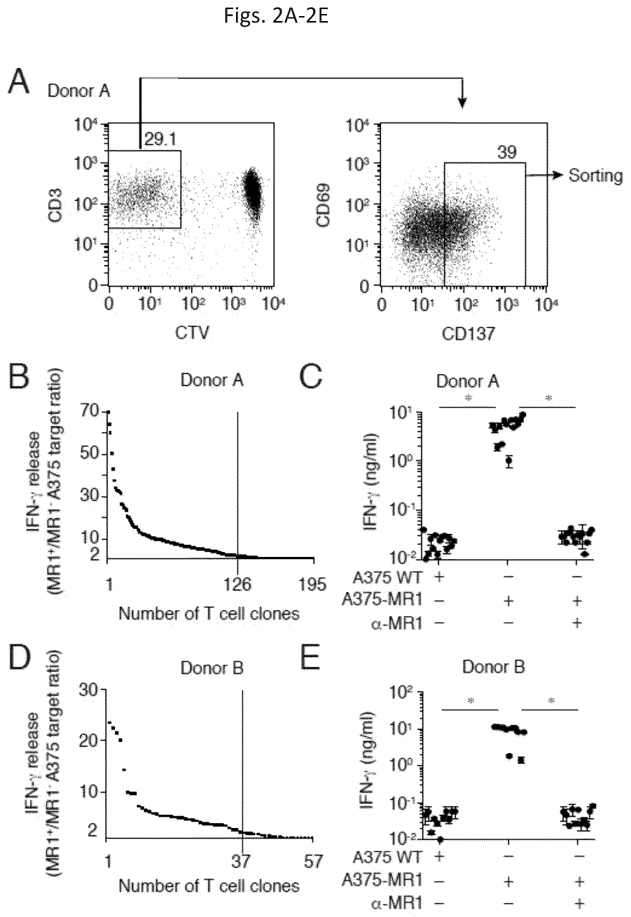

The inventors asked whether the DGB129 clone was representative of a novel population of tumour-reactive MR1-restricted T cells different from microbe-reactive MAIT cells. They therefore established a method to isolate and study these unpredicted MR1-restricted T cells. Purified T cells from two healthy donors were labelled with the proliferation marker CellTrace violet (CTV) and stimulated with irradiated A375-MR1 cells in the absence of exogenous antigens. Proliferating cells were re-challenged with A375-MR1 cells and those expressing high levels of the activation marker CD137 were sorted and cloned by limiting dilution (FIG. 2A). Individual T cell clones were then interrogated for their capacity to recognize A375-MR1 and A375 cells lacking MR1 (A375-WT). In both donors the inventors found that a major fraction of T cell clones (126/195 and 37/57, respectively) displayed specific recognition of A375-MR1 cells (FIG. 2B,D), which was inhibited by anti-MR1 blocking mAbs (FIG. 2C,E). Staining with TCR Vβ-specific mAbs of 12 MR1-reactive T cell clones revealed that they expressed 7 different TRBV chains (TRBV4-3, 6-5/6-6/6-9, 9, 18, 25-1, 28, 29-1) with some of the clones sharing the same TRBV gene. Furthermore, none expressed the TRAV1-2 chain, canonical for MAIT cells.

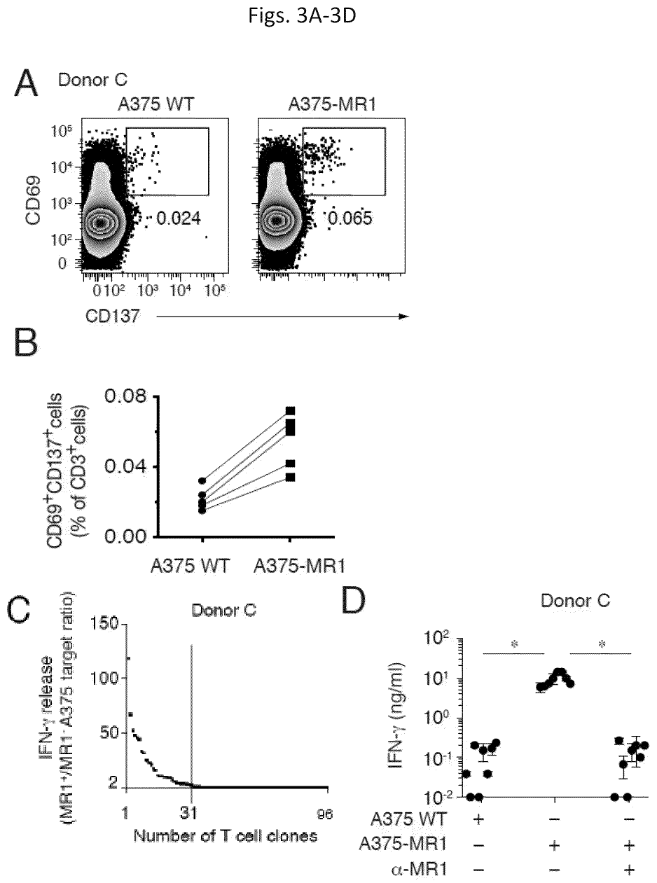

Lack of specific markers did not allow univocal identification of these novel T cells ex vivo by standard flow cytometry. Therefore, their frequency was estimated by combining flow cytometry analysis after very short-time in vitro stimulation and single T cell cloning experiments. Purified blood T cells from five healthy donors were co-cultured overnight with MR1-deficient or MR1-sufficient A375 cells and analysed for the expression of the activation markers CD69 and CD137 (FIG. 3A). In all of the five donors screened, the percentage of CD69⁺CD137⁺ T cells detected was consistently higher after stimulation with A375-MR1 cells (range 0.034-0.072% of T cells) than after co-culture with A375-WT cells (range 0.015-0.032%) (FIG. 3A,B). As the two types of APCs differed for MR1 expression, MR1-reactive T cells accounted for the increased numbers of activated T cells after stimulation with MR1-positive APCs. Using this approach, the inventors estimated that the circulating T cell pool of the analysed individuals contained A375-MR1-reactive T cells at frequency ranging between 1:2,500 (0.072-0.032=0.04%) and 1:5,000 (0.034-0.015=0.019%). This estimated frequency is higher than the frequency of peptide-specific CD4⁺ T cells after antigen exposure (Lucas et al., J Virol 78:7284-7287; Su et al., Immunity 38:373-383). These observations were supported by parallel experiments in which sorted CD69⁺CD137⁺ overnight-activated T cells from one of these donors (Donor C, FIG. 3A, right panel) were cloned. Indeed, 31 out of 96 screened T cell clones (32%) displayed specific reactivity to A375-MR1 cells (FIG. 3C), which was inhibited by anti-MR1 mAbs (FIG. 3D). Accordingly, the calculated frequency of A375-MR1-responsive T cells among blood T cells of this donor was 1:5,000 (0.065×0.32=0.02%), a value consistent with the estimated range. Detailed analysis of representative T cell clones derived from three donors confirmed that they displayed diverse TCRα and β chains and indicated differential expression of CD4, CD8 and CD161 (Table 1).

Collectively, these findings suggested that the identified tumour-reactive MR1-restricted T cells are a novel yet common polyclonal population of lymphocytes in the blood of healthy human individuals (hereafter termed MR1T cells).

### MR1T Cell TCR Gene Transfer Confers MR1-Restricted Recognition of Tumour Cells

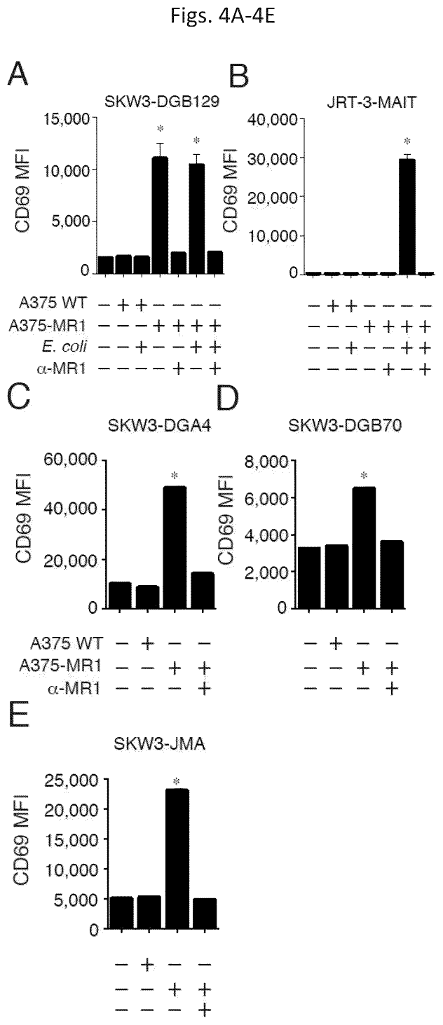

The inventors next investigated whether MR1T cell reactivity to tumour cells was mediated by the TCR. Expression of paired TCRα and β genes cloned from different MR1T cell clones in the TCR-deficient SKW-3 cells, conferred MR1 recognition of tumour cells which was comparable to that displayed by the original MR1T cells and which was completely blocked by anti-MR1-mAbs (FIG. 4A-C). In control experiments, transfer of TCRα and β genes of a representative MAIT cell clone conferred the ability to recognize A375-MR1 cells in MR1-dependent manner only in the presence of E. coli antigens (FIG. 4D). These data highlighted the critical role of the TCR in mediating MR1T cell recognition of tumour cells and suggested that MR1T cell TCR gene transfer can effectively redirect the reactivity of selected T cells toward MR1-expressing tumour cells.

### Differential Recognition of Tumour Cells by MR1T Cell Clones

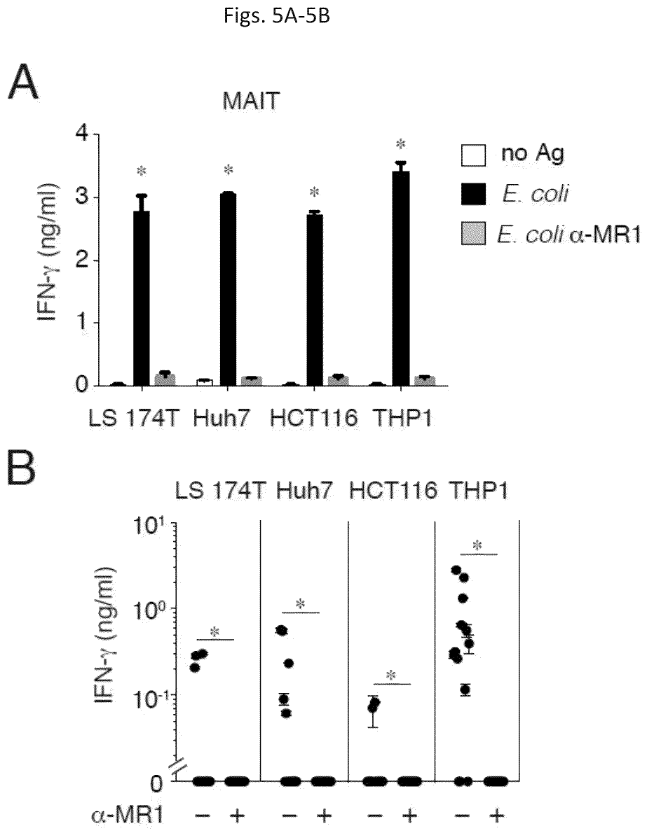

Having generated a large panel of MR1T cell clones reacting to MR1-expressing A375 melanoma cells, the inventors next investigated whether they could also recognize other types of tumour cells constitutively expressing surface MR1, including THP-1 myelomonocytic cells, Huh7 hepatoma cells, HCT116 colon carcinoma cells and LS 174T goblet-like colon adenocarcinoma cells. All these cell types supported MAIT cell activation in the presence of microbial antigens and in an MR1-dependent manner (FIG. 5A). The same cells were able to induce sterile activation of select MR1T cell clones to various extents. THP-1 cells were recognized by the majority of the tested MR1T cell clones, followed by the Huh7 hepatoma cells, the LS 174T goblet-like cells and the HCT116 colon carcinoma cells (FIG. 5B). Importantly, all responses were blocked by anti-MR1 mAbs.

These data further confirmed that MR1T cells are a novel and heterogeneous population of tumour-reactive T cells restricted to the non-polymorphic antigen-presenting molecule MR1.

### MR1T Cells Recognize MR1-Bound Antigens Present in Tumour Cells

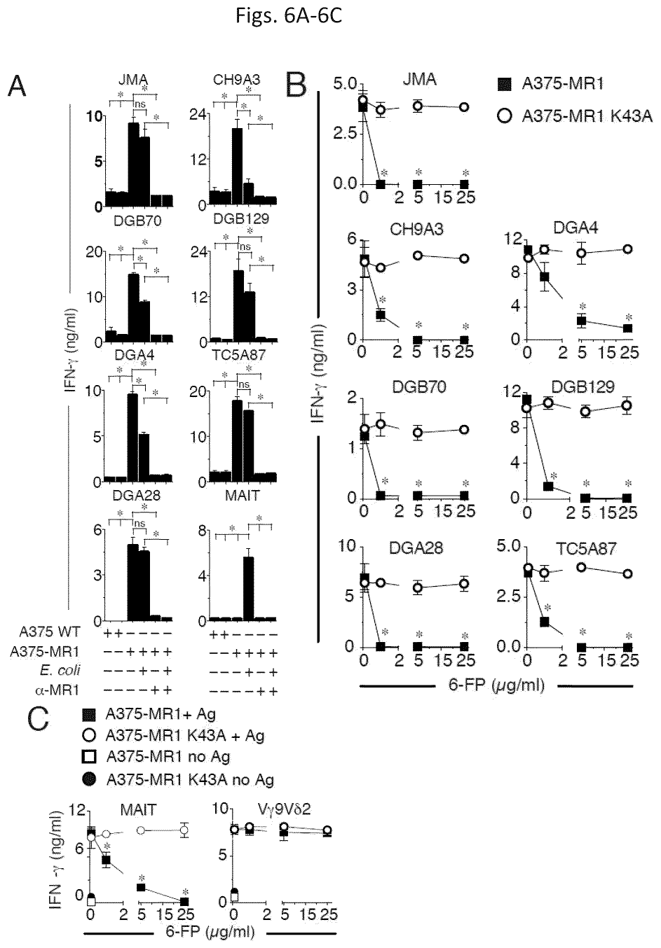

The inventors next studied the basis of MR1T cell reactivity to tumour cells. First, they sought to definitively rule out the possibility that MR1T cell clones could recognize microbial antigens, in analogy to MAIT cells. While a control MAIT cell clone reacted to A375-MR1 cells only in the presence of E. coli lysate, activation of different MR1T cell clones was not enhanced by the E. coli lysate (FIG. 6A). Consistent with these data, MR1-negative A375-WT cells failed to stimulate either type of T cells, irrespective of whether E. coli lysate was added, (FIG. 6A) and importantly anti-MR1 mAbs efficiently blocked both MR1T and MAIT cell responses (FIG. 6A). These findings confirmed that microbial ligands present in E. coli and stimulating MAIT cells do not stimulate the tested MR1T cells.

The inventors then tested the response of MR1T cells to the known MR1 ligands 6-FP and Ac-6-FP, which have previously been reported to stimulate a rare subset of TRAV1-2-negative T cells and inhibit MAIT cell activation by microbial antigens. MR1T cell stimulation was impaired in the presence of 6-FP or Ac-6-FP ligands, which also impaired E. coli stimulation of control MAIT cells, but did not disrupt control TCR yO cell responses to cognate antigen presented by the same APCs, thus excluding compound toxicity (FIG. 6B,C and 7A-C). Notably, 6-FP or Ac-6-FP failed to inhibit the activation of MR1T cells or MAIT cells when the target A375 cells were transduced to express mutant MR1 molecules with defective ligand binding capacity (blockade of Schiff base formation with ligands by mutation of Lysine 43 into Alanine, A375-MR1 K34A; FIG. 6B,C and 7D,E). The specific inhibition observed with 6-FP or Ac-6-FP indicated that MR1T cells i) do not recognize 6-FP and Ac-6-FP, ii) react to MR1-bound cellular antigens, and iii) are stimulated by ligands that do not require the formation of a Schiff base with MR1.

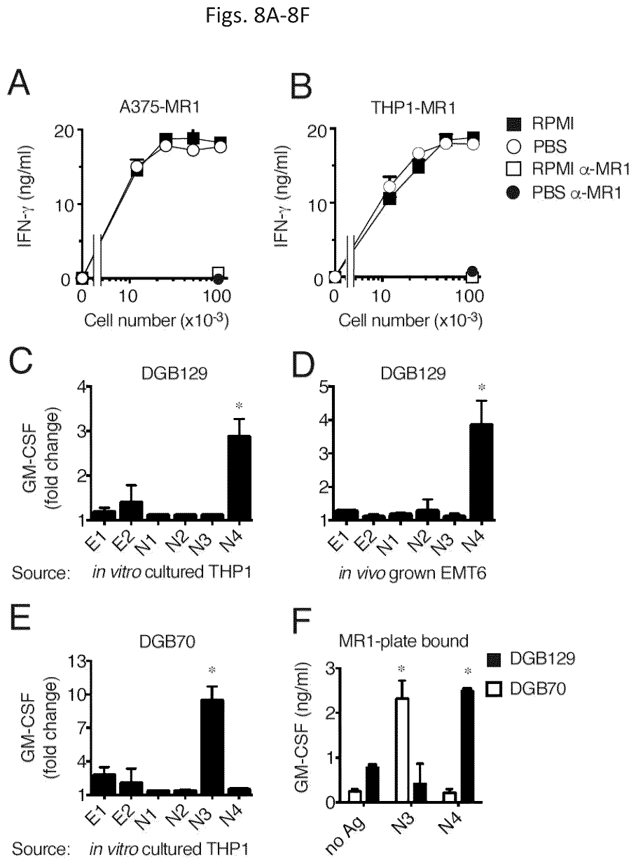

To gain further information on the origin of the recognized antigens the inventors asked whether the stimulatory capacity of tumour target cells was dependent on culture medium constituents, as some MR1 ligands, e.g. 6-FP, may derive from folate present in RPMI 1640 medium used for cell culture. Both THP-1 and A375-MR1 cells were extensively washed and cultivated 4 days in phosphate buffered saline solution (PBS) supplemented exclusively with 5% human serum. Cells were washed daily before being used to stimulate DGB129 MR1T cells and the T cell activation assays were performed in PBS. THP-1 and A375-MR1 cells grown in RPMI 1640 or in PBS showed the same stimulatory capacity (FIG. 8A,B), thus indicating that medium constituents are not responsible for MR1T cell activation. To directly investigate whether the stimulatory antigens were present in target tumour cells, the inventors then performed T cell activation assays using as source of antigen two types of tumour lysates. The first lysate was obtained from in vitro cultured THP-1 cells, while the second one was prepared from mouse breast tumours immediately after resection. Two hydrophobic and four hydrophilic fractions were obtained and tested using as APCs THP-1 cells that constitutively express low levels of MR1. The DGB129 clone reacted only to fraction N4, containing highly hydrophilic compounds isolated from both freshly explanted mouse tumour and in vitro cultured THP-1 cells (FIG. 8C,D). These results ruled out the possibility that stimulatory antigens were derived from RPMI 1640 components and indicated their cellular origin. The inventors also tested the fractions generated from THP-1 lysates with DGB70, another representative MR1T cell clone. DGB70 cells recognized fraction N3 and not N4, (FIG. 8E), suggesting that at least two distinct compounds differentially stimulated the two MR1T clones. The same fractions were also loaded onto plastic-bound MR1 molecules and showed alternative and specific stimulatory capacity, i.e. N3 stimulated only DGB70 cells, while N4 stimulated only DGB129 cells (FIG. 8F). In the absence of N3 and N4 fractions, the two clones did not react to MR1, further indicating the requirement of specific antigens.

In conclusion, these data indicated that MR1T cells recognize MR1 complexed with ligands not derived from culture medium and present also in tumour cells grown in vivo.

### MR1T Cells Display Differential Anti-Tumour Responses

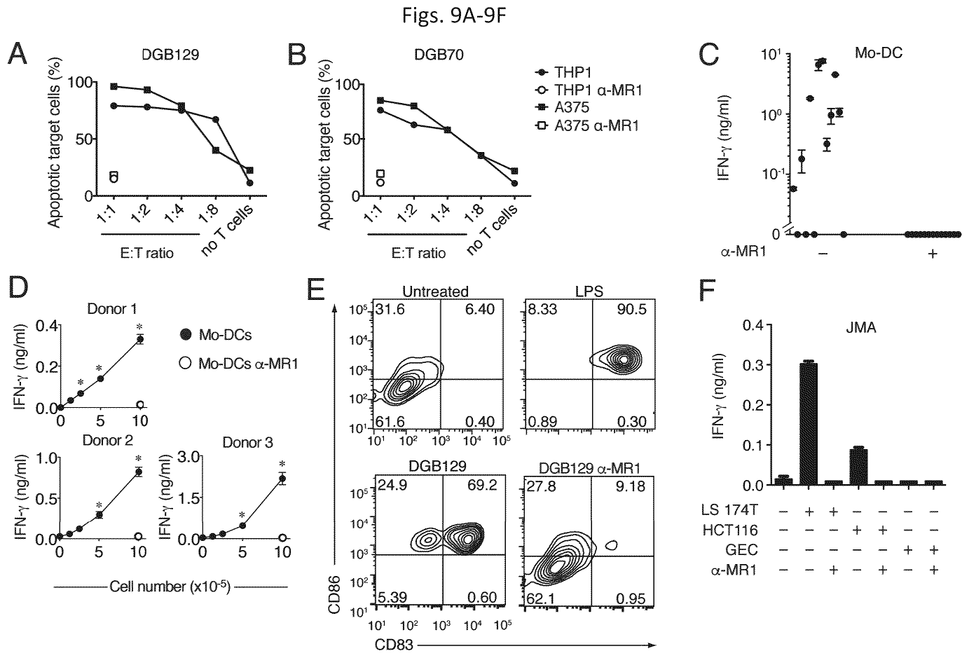

To assess the anti-tumour activity of MR1T cells the inventors tested their capacity to directly kill tumour cells in vitro. Two representative MR1T cell clones (DGB129 and DGB70) efficiently killed both MR1-expressing THP-1 and A375 cells at various effector:target ratios (FIG. 9A,B). A control MAIT cell clone failed to kill these two cell types, although it was fully capable of killing when targets were E. coli-infected (not shown). These results indicated that MR1T cells display specific cytotoxic activity against MR1-expressing tumour cells.

Having found that MR1 T cells recognized and killed the myelomonocytic tumour cell line THP-1, the inventors next addressed whether they could also recognize normal myeloid cells including monocytes and monocyte-derived dendritic cells (Mo-DC) from different donors. Monocytes were not recognized by any of the tested MR1T cell clones (not shown). By contrast, some MR1T cell clones reacted to Mo-DC in MR1 dependent manner (FIG. 9C). Interestingly, experiments performed with the representative DGB129 MR1T cell clone revealed that recognition of Mo-DC did not result in Mo-DC killing (not shown), but promoted up-regulation of CD83 and CD86 activation markers by Mo-DC (FIG. 9D). Remarkably, the activation of Mo-DC induced by DGB129 cells was fully inhibited by anti-MR1 mAbs (FIG. 9D). These data suggested that some tumour-reactive MR1T cells elicit direct anti-tumour activity and also promote activation of innate immune cells, with important implications in the establishment of effective anti-tumour immune responses.

As the inventors observed that some MR1T cell clones reacted to HCT116 and LS 174T intestinal tumour cells, they next investigated whether they could also recognize normal gut epithelial cells (GEC) prepared from gut biopsies. GEC cells were not stimulatory for any of the tested HCT116- or LS 174T-reactive MR1T cell clones (FIG. 9F,G), thus suggesting that MR1T cell clones may display specific recognition of gastrointestinal tumour cells while not reacting to normal intestinal epithelial cells.

To further assess the specificity of tumour recognition by MR1T cells, the inventors finally investigated whether they could react to other types of normal cells including neutrophils, NK cells, B cells and T cells. None of these cells were recognized by the tested MR1T cells (not shown).

Collectively, these data identify MR1T cells as a novel and heterogeneous population of human MR1-restricted T lymphocytes that i) differently react to various types of tumour cells, ii) display cytotoxic activity against tumour cells, iii) do not recognize normal cells with exception of in vitro-differentiated Mo-DC, and iv) do not kill Mo-DC but instead induce their activation. These findings suggested that MR1T cells display important anti-tumour properties and deserve to be exploited for their immunotherapeutic potential.

### MR1T Cells are Functionally Heterogeneous

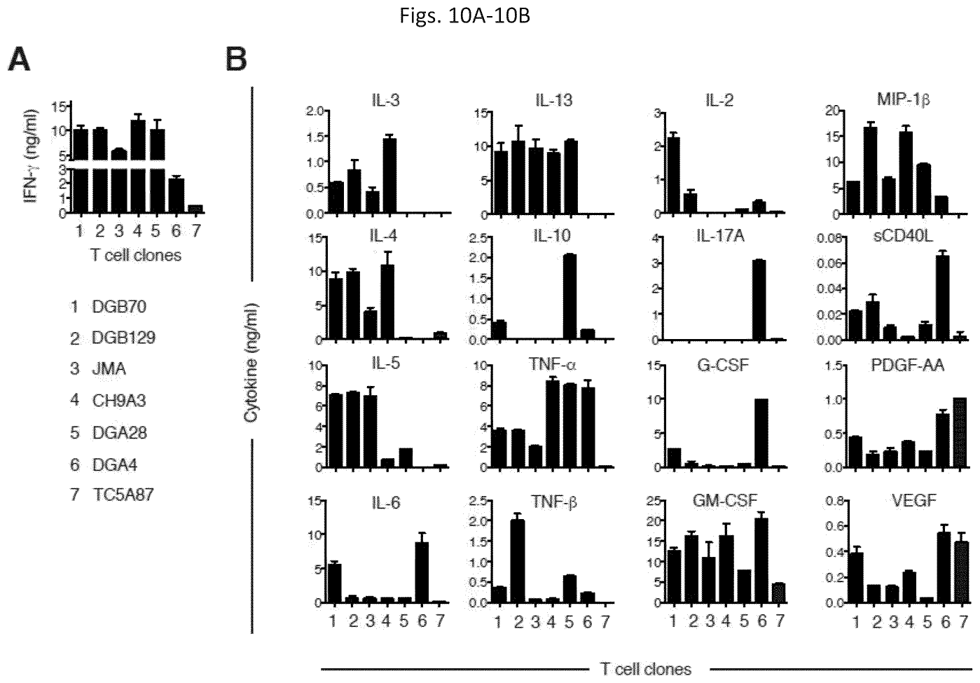

The inventors finally analyzed the cytokine secretion profile of representative MR1T cell clones upon stimulation by A375-MR1 tumour cells. All clones tested released IFN-γ (FIG. 10A). However, the inventors also observed diverse expression profiles of Th1 (IL-2, TNF-α and TNF-8), Th2 (IL-3, IL-4, IL-5, IL-6, IL-10, IL-13) and Th17 cytokines (IL-17A, G-CSF, GM-CSF), and other soluble factors (MIP-18, soluble CD40L PDGF-AA and VEGF; FIG. 10B). The variable combinations and quantities of cytokines expressed by MR1T cells suggested considerable functional plasticity within this population. For example, clone DGA4 secreted large quantities of IL-17A, IL-6, TNF-α and GM-CSF, but failed to secrete the prototypic Th2 cytokines IL-4, IL-5, IL-10 or IL-13, and thus displayed an ‘atypical’ Th17-like phenotype. In contrast, clone TC5A87 released substantial amounts of VEGF and PGDF-AA, but only little Th1 or Th2 cytokines, and no IL-17A. Notably, four of the seven clones studied (DGB129, CH9A3, DGB70, JMA) displayed a Th2-skewed profile of cytokine release, a functional phenotype which has been recently associated with protective anti-tumour immunity.

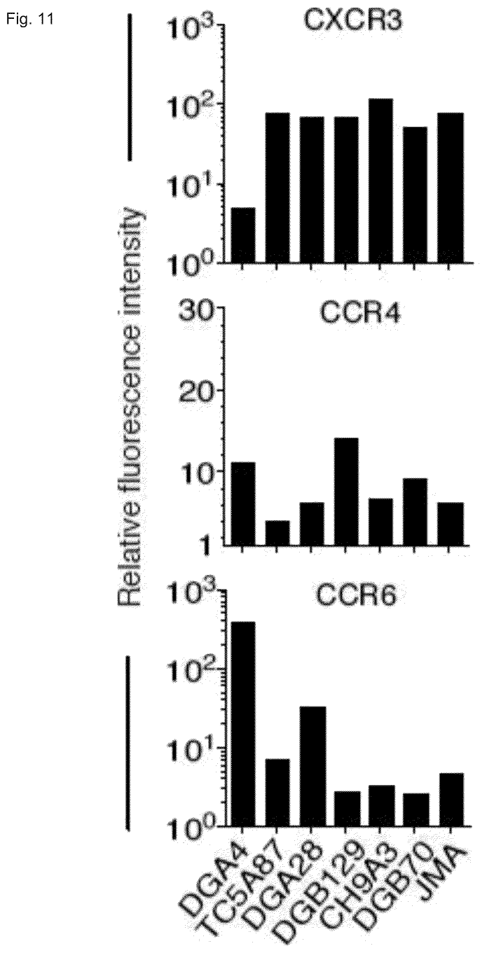

The inventors next investigated the expression of three selected chemokine receptors known to be differentially expressed by T cell subsets with distinct functions and whose alternative combined expression regulates T cell recirculation and migration to diverse homing sites. All MR1T cell clones but DGA4 displayed high levels of CXCR3 (FIG. 11). In addition, the inventors observed divergent expression patterns of CCR4 and CCR6 (FIG. 11), which further suggested that MR1T cells are heterogeneous.

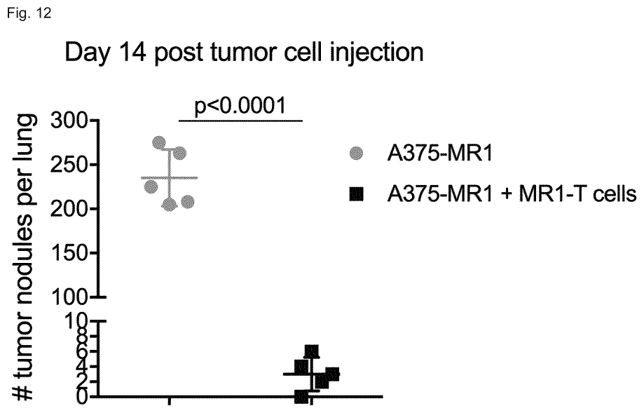

In a final series of studies it was investigated whether MR1T cells maintain their tumour-killing capacity in vivo using a lung solid tumour model. Mice intravenously injected with A375 melanoma cells expressing MR1 received DGB129 cells or were left untreated. On day 14, mice were sacrificed and the number of tumour nodules in the lungs was counted. While untreated mice showed 200-250 nodules, those treated with MR1T cells showed 1-6 nodules (FIG. 12). These results confirmed that in vivo growing tumour cells produce the antigens stimulating MR1T cells. Importantly, they provided strong evidence of the efficient capacity of MR1T cells to kill solid tumour cells in vivo.

Taken together, these data indicated that the tumour MR1-reactive T clones tested here are phenotypically and functionally diverse, thus suggesting that MR1T cells include multiple subsets with distinct recirculation patterns and tissue homing capacity and likely different roles in tumour immunity. In conclusion, these data identify MR1T cells as a novel population of human T lymphocytes that recognize MR1:tumour-associated-antigen complexes and may participate in anti-tumour immune responses with multiple effector functions.

TABLE 1
Phenotype of select MR1-reactive T cell clones.

| Clone | CD4 | CD8a | CD161 | TCRß |
|---|---|---|---|---|
| DGB129 | − | + | − | TRBV12-4 |
| DGB70 | − | − | − | TRBV28 |
| DGA28 | − | + | + | TRBV29-1 |
| DGA4 | − | − | + | TRBV6-1 |
| JMA | − | + | − | TRBV25-1 |
| TC5A87 | − | + | − | TRBV25-1 |
| CH9A3 | − | + | − | TRBV5-5 |

TABLE 2
Tumour cell lines recognized by human MR1T cells.

| Cell line | Origin |
|---|---|
| A375 | Human melanoma |
| CCRF-SB | Human B lymphoblastic leukemia |
| Huh7 | Human hepatocellular carcinoma |
| HCT116 | Human colon carcinoma |
| LS 174T | Human colon adenocarcinoma |
| THP-1 | Human myelomonocytic leukemia |

The following examples further illustrate the clinical workflow in which the invention is applied:

### Screening of MR1-Expressing Cancers

A cancer patient's tissue fresh or fresh-frozen tissue biopsies are analyzed for MR1 expression using mAbs specific for human MR1 and PCR amplification of MR1 mRNA.

### Cancer Therapy, Example 1: Selection of Best MRT1 TCR Genes for Recognition of Primary MR1-Expressing Cancer Cells

I. Primary MR1+ cancer cells isolated ex vivo are used to stimulate a library of previously characterized MR1T cell clones. Each clone expresses different TCR genes and recognizes different types of cancer cells.
II. The MR1T cells clones best responding to the cancer cells of the patient are selected and their TCR genes are used for TCR gene therapy. Response is assayed as a function of cytokine release and/or surface marker expression. Cells are assayed by internal (cytokine) or surface marker staining with antibodies reactive to the assayed activation markers, exemplified but not restricted to CD3, CD69, CD137, CD150, and/or ICOS (surface markers) and INF-γ and GM-CSF (cytokine).
III. When available soluble MR1T TCR will be multimerized and used to stain tumor cells isolated from tumour biopsies. The MR1T TCR multimers binding to tumour cells will allow rapid selection of MR1T TCRs suitable for gene therapy in that patient.
IV. Several circulating patient T cell populations may be used as recipient T cells (naïve, central memory, effector memory, CD4+, CD8+, or CD4, CD8 double negative T cells). Naïve T cells are selected to allow unprimed T lymphocytes to mature in the presence of tumor cells when they are transduced with TCR genes recognizing MR1-tumor antigens. Central and effector memory cells are used because they provide immediate proliferation and effector functions (tumor killing) upon recognition of tumor cells expressing MR1. CD4 cells are selected to provide sufficient numbers of T helper cells that facilitate recruitment and expansion of other cells with anti-tumor functions. CD8 T cells are selected to facilitate killing of tumor cells. CD4-CD8 double negative T cells are selected for their innate-like functions such as immediate release of large amounts of killer effector molecules (TNFα, granzymes and granulysin).
V. T cells expressing the transduced TCR genes and with selected effector functions are used for adoptive cell therapy (ACT).
   T cells from peripheral blood of patients are stained with monoclonal antibodies specific for surface markers (CD4, CD8, CD27, CD45RA, CD57) and sorted. Each sorted population is activated with Dynabeads® Human T-Activator CD3/CD28 (ThermoFisher) and 24 h later transfected with the TCR genes encoding the MR1T TCR selected for the individual patient. This yields a modified T cell preparation (recipient T cells). In some cases, recipient T cells are also modified by gene-editing methods to inactivate PD1, ILT2 and ILT4 inhibitory genes or were transduced with CD137 and CD134 genes to promote cell survival, cell expansion and to enhance anti-cancer effector function.
   Lymphodepletion is made in recipient cancer patients using a non-myeloablative chemotherapy preparative regimen (60 mg/kg cyclophosphamide for 2 days and 25 mg/m 2 fludarabine administered for 5 days) followed by transfer of T cells and IL-2 given at 720,000 IU/kg to tolerance. In some instances, 200 or 1200 centigray (cGy; 1 Gy=100 rads) total-body irradiation is added to the preparative regimen. T cells expressing the MR1T exogenous TCR genes (the modified T cell preparation) are transferred into recipient.
   TCR genes are cloned in safe recombinant lentivirus vectors (see for example Provasi et al., Nat Med 18, 807-815 (2012)), which contain suicide genes and cannot produce mature viral particles in the absence of other helper viruses. In some cases, TCR genes are cloned in vectors containing suicide genes (for examples, see Greco et al., Front Pharmacol 6, 95 (2015)), thus reducing the risks derived from unwanted gene insertion. In some cases RNA encoding the TCR MR1T genes is transfected in recipient cells (see for example Zhao et al. Molecular therapy 13, 151, 2006)).

### Cancer Therapy, Example 2: Isolation of MR1T Cells from Tumor-Infiltrating Lymphocytes (TILs) of Patient to be Treated

VI. Autologous TILs are prepared from the cancer tissue biopsies according to our previously established protocol (De Libero, ibid.).
VII. T cells are expanded in vitro for 2-3 weeks using medium supplemented with IL-2, IL-7, and IL-15.
VIII. Expanded T cells are tested for reactivity against autologous MR1⁺ cancer cells. T cells that increase surface expression of activation markers (CD137, CD150, CD69, ICOS) are considered cancer-specific and if they are inhibited by the presence of anti-MR1 monoclonal antibodies, they are considered MR1-dependent.
   Cancer-reactive T cells are sorted according to the expression of one of above activation markers and expanded and used for ACT, as outlined above.

TABLE
designation of sequence ID NOs

| | Protein | | | | | |
|---|---|---|---|---|---|---|
| | | | α | β | Nucleic acid | |
| Clone | α | β | CDR3 | CDR3 | α | β |
| 1 | 1 | 2 | 65 | 80 | 7 | 8 |
| 2 | 3 | 4 | 66 | 81 | 9 | 10 |
| 3 | 5 | 6 | 67 | 82 | 11 | 12 |
| 4 | 13 | 25 | 68 | 83 | 37 | 49 |
| 5 | 14 | 26 | 69 | 84 | 38 | 50 |
| 6 | 15 | 27 | 70 | 85 | 39 | 51 |
| 7 | 16 | 28 | 71 | 86 | 40 | 52 |
| 8 | 17 | 29 | 72 | 87 | 41 | 53 |
| 9 | 18 | 30 | 73 | 88 | 42 | 54 |
| 10 | 19 | 31 | 74 | 89 | 43 | 55 |
| 11 | 20 | 32 | 75 | 90 | 44 | 56 |
| 12 | 21 | 33 | 76 | 91 | 45 | 57 |
| 13 | 22 | 34 | 77 | 92 | 46 | 58 |
| 14 | 23 | 35 | 78 | 93 | 47 | 59 |
| 15 | 24 | 36 | 79 | 94 | 48 | 60 |
| Clone | γ | δ | γCDR3 | δCDR3 | γ | δ |
| (1) | 61 | 62 | 95 | 96 | 63 | 64 |

Sequences
Variable domains and CDR3 of TCR chain sequences of the invention. CDR3 domains
annotated below according to the IMGT system as assigned by the Domain Gap Align tool
version 4.10.3 provided by Ehrenmann et al. Nucleic Acids Res. 38: D301-D307 2010. As
indicated, variable domains of the following sequences are underlined, noted CDR sequences
are in bold.

SEQ ID NO 001: TCR alpha chain clone 1 (MCA2E7), variable domain, [symbol]
MAMLLGASVLILWLQPDWVNSQQKNDDQQVKQNSPSLSVQEGRISILNCDYTNSMFDYFL
WYKKYPAEGPTFLISISSIKDKNEDGRFTVFLNKSAKHLSLHIVPSQPGDSAVYFC [symbol] [symbol]
FGQGTELSVKPNIQNPDPAVYQLRDSKSSDKSVCLFTDFDSQTNVSQSKDSDVYITD
KTVLDMRSMDFKSNSAVAWSNKSDFACANAFNNSIIPEDTFFPSPESSCDVKLVEKSFETDT
NLNFQNLSVIGFRILLLKVAGFNLLMTLRLWSS
SEQ ID NO 002: TCR beta chain clone 1 (MCA2E7), variable domain, [symbol]
MGIRLLCRVAFCFLAVGLVDVKVTQSSRYLVKRTGEKVFLECVQDMDHENMFWYRQDPGL
GLRLIYFSYDVKMKEKGDIPEGYSVSREKKERFSLILESASTNQTSMYLC [symbol] FG
PGTRLTVTEDLKNVFPPEVAVFEPSEAEISHTQKATLVCLATGFYPDHVELSWWVNGKEVH
SGVSTDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMAMVKR
KDSRG
SEQ ID NO 003: TCR alpha chain clone 2 (MCA3C3), variable domain, [symbol]
MISLRVLLVILWLQLSWVWSQRKEVEQDPGPFNVPEGATVAFNCTYSNSASQSFFWYRQD
CRKEPKLLMSVYSSGNEDGRFTAQLNRASQYISLLIRDSKLSDSATYLCV [symbol] FGTGT
SLTVIPNIQNPDPAVYQLRDSKSSDKSVCLFTDFDSQTNVSQSKDSDVYITDKTVLDMRSMD
FKSNSAVAWSNKSDFACANAFNNSIIPEDTFFPSPESSCDVKLVEKSFETDTNLNFQNLSVI
GFRILLLKVAGFNLLMTLRLWSS
SEQ ID NO 004: TCR beta chain clone 2 (MCA3C3), variable domain, [symbol]
MASLLFFCGAFYLLGTGSMDADVTQTPRNRITKTGKRIMLECSQTKGHDRMYWYRQDPGL
GLRLIYYSFDVKDINKGEISDGYSVSRQAQAKFSLSLESAIPNQTALYFC [symbol] [symbol]
FGEGSRLTVLEDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQFYGLSENDEW
TQDRAKPVTQIVSAEAWGRADCGFTSESYQQGVLSATILYEILLGKATLYAVLVSALVLMAM
VKRKDSRG
SEQ ID NO 005: TCR alpha chain clone 3 (CHO9A4), variable domain, [symbol]
MLTASLLRAVIASICVVSSMAQKVTQAQTEISVVEKEDVTLDCVYETRDTTYYLFWYKQPPS
GELVFLIRRNSFDEQNEISGRYSWNFQKSTSSFNFTITASQVVDSAVYFC [symbol] [symbol]
FGQGTTLQVKPDIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLVEKSFETDTNLN
FQNLSVIGFRILLLKVAGFNLLMTLRLWSS
SEQ ID NO 006: TCR beta chain clone 3 (CHO9A4), variable domain, [symbol]
MGIRLLCRVAFCFLAVGLVDVKVTQSSRYLVKRTGEKVFLECVQDMDHENMFWYRQDPGL
GLRLIYFSYDVKMKEKGDIPEGYSVSREKKERFSLILESASTNQTSMYLC [symbol] [symbol]
FGPGTRLTVLEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQFYGLSENDE
WTQDRAKPVTQIVSAEAWGRADCGFTSESYQQGVLSATILYEILLGKATLYAVLVSALVLMA
MVKRKDSRG
SEQ ID NO 007:
Atggccatgctcctgggggcatcagtgctgattctgtggcttcagccagactgggtaaacagtcaacagaagaatgatgaccag
caagttaagcaaaattcaccatccctgagcgtccaggaaggaagaatttctattctgaactgtgactatactaacagcatgtttgatt
atttcctatggtacaaaaaataccctgctgaaggtcctacattcctgatatctataagttccattaaggataaaaatgaagatggaag
attcactgtcttcttaaacaaaagtgccaagcacctctctctgcacattgtgccctcccagcctggagactctgcagtgtacttctgtgc
agcccagatttataaccagggaggaaagcttatcttcggacagggaacggagttatctgtgaaacccaatatccagaaccctga
ccctgccgtgtaccagctgagagactctaaatccagtgacaagtctgtctgcctattcaccgattttgattctcaaacaaatgtgtcac
aaagtaaggattctgatgtgtatatcacagacaaaactgtgctagacatgaggtctatggacttcaagagcaacagtgctgtggcc
tggagcaacaaatctgactttgcatgtgcaaacgccttcaacaacagcattattccagaagacaccttcttccccagcccagaaa
gttcctgtgatgtcaagctggtcgagaaaagctttgaaacagatacgaacctaaactttcaaaacctgtcagtgattgggttccgaa
tcctcctcctgaaagtggccgggtttaatctgctcatgacgctgcggctgtggtccagc
SEQ ID NO 008:
atgggaatcaggctcctgtgtcgtgtggccttttgtttcctggctgtaggcctcgtagatgtgaaagtaacccagagctcgagatatct
agtcaaaaggacgggagagaaagtttttctggaatgtgtccaggatatggaccatgaaaatatgttctggtatcgacaagaccca
ggtctggggctacggctgatctatttctcatatgatgttaaaatgaaagaaaaaggagatattcctgaggggtacagtgtctctaga
gagaagaaggagcgcttctccctgattctggagtccgccagcaccaaccagacatctatgtacctctgtgccagcagtttttctagc
ggaaagcagtacttcgggccgggcaccaggctcacggtcacagaggacctgaaaaacgtgttcccacccgaggtcgctgtgttt
gagccatcagaagcagagatctcccacacccaaaaggccacactggtgtgcctggccacaggcttctaccccgaccacgtgg
agctgagctggtgggtgaatgggaaggaggtgcacagtggggtcagcacagacccgcagcccctcaaggagcagcccgcc
ctcaatgactccagatactgcctgagcagccgcctgagggtctcggccaccttctggcagaacccccgcaaccacttccgctgtc
aagtccagttctacgggctctcggagaatgacgagtggacccaggatagggccaaacctgtcacccagatcgtcagcgccgag
gcctggggtagagcagactgtggcttcacctccgagtcttaccagcaaggggtcctgtctgccaccatcctctatgagatcttgcta
gggaaggccaccttgtatgccgtgctggtcagtgccctcgtgctgatggccatggtcaagagaaaggattccagaggc
SEQ ID NO 009:
atgatatccttgagagttttactggtgatcctgtggcttcagttaagctgggtttggagccaacggaaggaggtggagcaggatcctg
gacccttcaatgttccagagggagccactgtcgctttcaactgtacttacagcaacagtgcttctcagtctttcttctggtacagacag
gattgcaggaaagaacctaagttgctgatgtcggtatactccagtggtaatgaagatggaaggtttacagcacagctcaatagag
ccagccagtatatttccctgctcatcagagactccaagctcagtgattcagccacctacctctgtgtggtgaccggtaaccagttcta
ttttgggacagggacaagtttgacggtcattccaaatatccagaaccctgaccctgccgtgtaccagctgagagactctaaatcca
gtgacaagtctgtctgcctattcaccgattttgattctcaaacaaatgtgtcacaaagtaaggattctgatgtgtatatcacagacaaa
actgtgctagacatgaggtctatggacttcaagagcaacagtgctgtggcctggagcaacaaatctgactttgcatgtgcaaacgc
cttcaacaacagcattattccagaagacaccttcttccccagcccagaaagttcctgtgatgtcaagctggtcgagaaaagctttga
aacagatacgaacctaaactttcaaaacctgtcagtgattgggttccgaatcctcctcctgaaagtggccgggtttaatctgctcatg
acgctgcggctgtggtccagc
SEQ ID NO 010:
atggcctccctgctcttcttctgtggggccttttatctcctgggaacagggtccatggatgctgatgttacccagaccccaaggaatag
gatcacaaagacaggaaagaggattatgctggaatgttctcagactaagggtcatgatagaatgtactggtatcgacaagaccc
aggactgggcctacggttgatctattactcctttgatgtcaaagatataaacaaaggagagatctctgatggatacagtgtctctcga
caggcacaggctaaattctccctgtccctagagtctgccatccccaaccagacagctctttacttctgtgccaccagtgatgtcggg
acaggggacaccggggagctgttttttggagaaggctctaggctgaccgtactggaggacctgaaaaacgtgttcccacccgag
gtcgctgtgtttgagccatcagaagcagagatctcccacacccaaaaggccacactggtgtgcctggccacaggcttctaccccg
accacgtggagctgagctggtgggtgaatgggaaggaggtgcacagtggggtcagcacagacccgcagcccctcaaggagc
agcccgccctcaatgactccagatactgcctgagcagccgcctgagggtctcggccaccttctggcagaacccccgcaaccact
tccgctgtcaagtccagttctacgggctctcggagaatgacgagtggacccaggatagggccaaacctgtcacccagatcgtca
gcgccgaggcctggggtagagcagactgtggcttcacctccgagtcttaccagcaaggggtcctgtctgccaccatcctctatga
gatcttgctagggaaggccaccttgtatgccgtgctggtcagtgccctcgtgctgatggccatggtcaagagaaaggattccagag
gc
SEQ ID NO 011:
atgctgactgccagcctgttgagggcagtcatagcctccatctgtgttgtatccagcatggctcagaaggtaactcaagcgcagac
tgaaatttctgtggtggagaaggaggatgtgaccttggactgtgtgtatgaaacccgtgatactacttattacttattctggtacaagc
aaccaccaagtggagaattggttttccttattcgtcggaactcttttgatgagcaaaatgaaataagtggtcggtattcttggaacttcc
agaaatccaccagttccttcaacttcaccatcacagcctcacaagtcgtggactcagcagtatacttctgtgctctgagtgaggaac
ctagcaacacaggcaaactaatctttgggcaagggacaactttacaagtaaaaccagatatccagaaccctgaccctgccgtgt
accagctgagagactctaaatccagtgacaagtctgtctgcctattcaccgattttgattctcaaacaaatgtgtcacaaagtaagg
attctgatgtgtatatcacagacaaaactgtgctagacatgaggtctatggacttcaagagcaacagtgctgtggcctggagcaac
aaatctgactttgcatgtgcaaacgccttcaacaacagcattattccagaagacaccttcttccccagcccagaaagttcctgtgat
gtcaagctggtcgagaaaagctttgaaacagatacgaacctaaactttcaaaacctgtcagtgattgggttccgaatcctcctcctg
aaagtggccgggtttaatctgctcatgacgctgcggctgtggtccagc
SEQ ID NO 012:
atgggaatcaggctcctgtgtcgtgtggccttttgtttcctggctgtaggcctcgtagatgtgaaagtaacccagagctcgagatatct
agtcaaaaggacgggagagaaagtttttctggaatgtgtccaggatatggaccatgaaaatatgttctggtatcgacaagaccca
ggtctggggctacggctgatctatttctcatatgatgttaaaatgaaagaaaaaggagatattcctgaggggtacagtgtctctaga
gagaagaaggagcgcttctccctgattctggagtccgccagcaccaaccagacatctatgtacctctgtgccagcagtcgactac
tagcgggggggcagaatgagcagttcttcgggccagggacacggctcaccgtgctagaggacctgaaaaacgtgttcccacc
cgaggtcgctgtgtttgagccatcagaagcagagatctcccacacccaaaaggccacactggtatgcctggccacaggcttcta
ccccgaccacgtggagctgagctggtgggtgaatgggaaggaggtgcacagtggggtcagcacagacccgcagcccctcaa
ggagcagcccgccctcaatgactccagatactgcctgagcagccgcctgagggtctcggccaccttctggcagaacccccgca
accacttccgctgtcaagtccagttctacgggctctcggagaatgacgagtggacccaggatagggccaaacccgtcacccag
atcgtcagcgccgaggcctggggtagagcagactgtggcttcacctccgagtcttaccagcaaggggtcctgtctgccaccatcc
tctatgagatcttgctagggaaggccaccttgtatgccgtgctggtcagtgccctcgtgctgatggccatggtcaagagaaaggatt
ccagaggc
SEQ ID NO 013: TCR alpha chain clone 4 (DGA4), variable domain, [symbol]
MWGVFLLYVSMKMGGTTGQNIDQPTEMTATEGAIVQINCTYQTSGFNGLFWYQQHAGEAP
TFLSYNVLDGLEEKGRFSSFLSRSKGYSYLLLKELQMKDSASYLC [symbol] FGSGTRL
LVRPDIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSCDVKLVEKSFETDTNLNFQNLSVIG
FRILLLKVAGFNLLMTLRLWSS
SEQ ID NO 014: TCR alpha chain clone 5 (DGA28), variable domain, [symbol]
MLLITSMLVLWMQLSQVNGQQVMQIPQYQHVQEGEDFTTYCNSSTTLSNIQWYKQRPGGH
PVFLIQLVKSGEVKKQKRLTFQFGEAKKNSSLHITATQTTDVGTYFC [symbol] FGQ
GTILTVHPNIQNPDPAVYQLRDSKSSDKSVCLFTDFDSQTNVSQSKDSDVYITDKTVLDMRS
MDFKSNSAVAWSNKSDFACANAFNNSIIPEDTFFPSPESSCDVKLVEKSFETDTNLNFQNLS
VIGFRILLLKVAGFNLLMTLRLWSS
SEQ ID NO 015: TCR alpha chain clone 6 (DGB70), variable domain, [symbol]
MKTFAGFSFLFLWLQLDCMSRGEDVEQSLFLSVREGDSSVINCTYTDSSSTYLYWYKQEPG
AGLQLLTYIFSNMDMKQDQRLTVLLNKKDKHLSLRIADTQTGDSAIYFC [symbol] [symbol]
FGRGTQLTVWPDIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTNL
NFQNLSVIGFRILLLKVAGFNLLMTLRLWSS
SEQ ID NO 016: TCR alpha chain clone 7 (DGB129), variable domain, [symbol]
MAMLLGASVLILWLQTDWVNSQQKNDDQQVKQNSPSLSVQEGRISILNCDYTNSMFDYFL
WYKKYPAEGPTFLISISSIKDKNEDGRFTVFLNKSAKHLSLHIVPSQPGDSAVYFC [symbol] [symbol]
FGQGTELSVKPNIQNPDPAVYQLRDSKSSDKSVCLFTDFDSQTNVSQSKDSDVYITD
KTVLDMRSMDFKSNSAVAWSNKSDFACANAFNNSIIPEDTFFPSPESSCDVKLVEKSFETDT
NLNFQNLSVIGFRILLLKVAGFNLLMTLRLWSS
SEQ ID NO 17: TCR alpha chain clone 8 (CH9A3), variable domain, [symbol]
MEKNPLAAPLLILWFHLDCVSSILNVEQSPQSLHVQEGDSTNFTCSFPSSNFYALHWYRWE
TAKSPEALFVMTLNGDEKKKGRISATLNTKEGYSYLYIKGSQPEDSATYLC [symbol] F
GKGTSLLVTPHIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
SEQ ID NO 018: TCR alpha chain clone 9 (JM64-8), variable domain, [symbol]
MNYSPGLVSLILLLLGRTRGNSVTQMEGPVTLSEEAFLTINCTYTATGYPSLFWYVQYPGEG
LQLLLKATKADDKGSNKGFEATYRKETTSFHLEKGSVQVSDSAVYFC [symbol] [symbol]
FGKGTKLSVKPNIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLVEKSFETDTNLN
FQNLSVIGFRILLLKVAGFNLLMTLRLWSS
SEQ ID NO 019: TCR alpha chain clone 10 (JMA), variable domain, [symbol]
MVLKFSVSILWIQLAWVSTQLLEQSPQFLSIQEGENLTVYCNSSSVFSSLQWYRQEPGEGP
VLLVTVVTGGEVKKLKRLTFQFGDARKDSSLHITAAQPGDTGLYLC [symbol] FGKGT
SLLVTPHIQNPDPAVYQLRDSKSSDKSVCLFTDFDSQTNVSQSKDSDVYITDKTVLDMRSM
DFKSNSAVAWSNKSDFACANAFNNSIIPEDTFFPSPESSCDVKLVEKSFETDTNLNFQNLSVI
GFRILLLKVAGFNLLMTLRLWSS
SEQ ID NO 020: TCR alpha chain clone 11 (LMB1F3), variable domain, [symbol]
MMKSLRVLLVILWLQLSWVWSQQKEVEQDPGPLSVPEGAIVSLNCTYSNSAFQYFMWYRQ
YSRKGPELLMYTYSSGNKEDGRFTAQVDKSSKYISLFIRDSQPSDSATYLC [symbol] [symbol]
FGRGTSLIVHPYIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLVEKSFETDTNLN
FQNLSVIGFRILLLKVAGFNLLMTLRLWSS
SEQ ID NO 021: TCR alpha chain clone 12 (MCA3D9), variable domain, [symbol]
MLLEHLLIILWMQLTWVSGQQLNQSPQSMFIQEGEDVSMNCTSSSIFNTWLWYKQDPGEG
PVLLIALYKAGELTSNGRLTAQFGITRKDSFLNISASIPSDVGIYFC [symbol]
FGQGTILTVHPNIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNF
QNLSVIGFRILLLKVAGFNLLMTLRLWSS
SEQ ID NO 022: TCR alpha chain clone 13 (TC5A87), variable domain, [symbol]
MTSIRAVFIFLWLQLDLVNGENVEQHPSTLSVQEGDSAVIKCTYSDSASNYFPWYKQELGK
GPQLIIDIRSNVGEKKDQRIAVTLNKTAKHFSLHITETQPEDSAVYFC [symbol] F
GTGTRLTIIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
SEQ ID NO 023: TCR alpha chain clone 14 (SMC3), variable domain, [symbol]
MWGVFLLYVSMKMGGTTGQNIDQPTEMTATEGAIVQINCTYQTSGFNGLFWYQQHAGEAP
TFLSYNVLDGLEEKGRFSSFLSRSKGYSYLLLKELQMKDSASYLC [symbol] WGAGTK
LIIKPDIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSCDVKLVEKSFETDTNLNFQNLSVIG
FRILLLKVAGFNLLMTLRLWSS
SEQ ID NO 024: TCR alpha chain clone 15 (TRA44), variable domain, [symbol]
MISLRVLLVILWLQLSWVWSQRKEVEQDPGPFNVPEGATVAFNCTYSNSASQSFFWYRQD
CRKEPKLLMSVYSSGNEDGRFTAQLNRASQYISLLIRDSKLSDSATYLC [symbol]
FGAGTILRVKSYIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNF
QNLSVIGFRILLLKVAGFNLLMTLRLWSS
SEQ ID NO 025: TCR beta chain clone 4 (DGA4), variable domain, [symbol]
MSIGLLCCVAFSLLWASPVNAGVTQTPKFQVLKTGQSMTLQCAQDMNHNSMYWYRQDPG
MGLRLIYYSASEGTTDKGEVPNGYNVSRLNKREFSLRLESAAPSQTSVYFC [symbol] [symbol]
FGPGTRLTVLEDLKNVFPPEVAVFEPSEAEISHTQKATLVCLATGFYPDHVELSWWVN
GKEVHSGVSTDPQPLKEQPALNDSRYCLSSRLRVSATFWQNPRNHFRCQVQFYGLSEND
EWTQDRAKPVTQIVSAEAWGRADCGFTSESYQQGVLSATILYEILLGKATLYAVLVSALVLM
AMVKRKDSRG
SEQ ID NO 026: TCR beta chain clone 5 (DGA28), variable domain, [symbol]
MLSLLLLLLGLGSVFSAVISQKPSRDICQRGTSLTIQCQVDSQVTMMFWYRQQPGQSLTLIA
TANQGSEATYESGFVIDKFPISRPNLTESTLTVSNMSPEDSSIYLCSVGAGQGPYTDTQYFG
PGTRLTVLEDLKNVFPPEVAVFEPSEAEISHTQKATLVCLATGFYPDHVELSWWVNGKEVH
SGVSTDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMAMVKR
KDSRG
SEQ ID NO 027: TCR beta chain clone 6 (DGB70), variable domain, [symbol]
MGIRLLCRVAFCFLAVGLVDVKVTQSSRYLVKRTGEKVFLECVQDMDHENMFWYRQDPGL
GLRLIYFSYDVKMKEKGDIPEGYSVSREKKERFSLILESASTNQTSMYLC [symbol] [symbol]
FGSGTQLSVLEDLNKVFPPEVAVFEPSEAEISHTQKATLVCLATGFFPDHVELSWWVNG
KEVHSGVSTDPQPLKEQPALNDSRYCLSSRLRVSATFWQNPRNHFRCQVQFYGLSENDE
WTQDRAKPVTQIVSAEAWGRADCGFTSVSYQQGVLSATILYEILLGKATLYAVLVSALVLMA
MVKRKDF
SEQ ID NO 028: TCR beta chain clone 7 (DGB129), variable domain, [symbol]
MGSWTLCCVSLCILVAKHTDAGVIQSPRHEVTEMGQEVTLRCKPISGHDYLFWYRQTMMR
GLELLIYFNNNVPIDDSGMPEDRFSAKMPNASFSTLKIQPSEPRDSAVYFC [symbol] F
GQGTRLTVVEDLNKVFPPEVAVFEPSEAEISHTQKATLVCLATGFFPDHVELSWWVNGKEV
HSGVSTDPQPLKEQPALNDSRYCLSSRLRVSATFWQNPRNHFRCQVQFYGLSENDEWTQ
DRAKPVTQIVSAEAWGRADCGFTSVSYQQGVLSATILYEILLGKATLYAVLVSALVLMAMVK
RKDF
SEQ ID NO 029: TCR beta chain clone 8 (CH9A3), variable domain, [symbol]
MGPGLLCWVLLCLLGAGPVDAGVTQSPTHLIKTRGQHVTLRCSPISGHKSVSWYQQVLGQ
GPQFIFQYYEKEERGRGNFPDRFSARQFPNYSSELNVNALLLGDSALYLC [symbol] [symbol]
FGNGTRLTVTEDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQFYGLSENDEW
TQDRAKPVTQIVSAEAWGRADCGFTSVSYQQGVLSATILYEILLGKATLYAVLVSALVLMAM
VKRKDF
SEQ ID NO 030: TCR beta chain clone 9 (JM64-8), variable domain, [symbol]
MGPGLLHWMALCLLGTGHGDAMVIQNPRYQVTQFGKPVTLSCSQTLNHNVMYWYQQKSS
QAPKLLFHYYDKDFNNEADTPDNFQSRRPNTSFCFLDIRSPGLGDAAMYLC [symbol] [symbol]
FGPGTRLLVLEDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQFYGLSENDEW
TQDRAKPVTQIVSAEA
SEQ ID NO 031: TCR beta chain clone 10 (JMA), variable domain, [symbol]
MTIRLLCYMGFYFLGAGLMEADIYQTPRYLVIGTGKKITLECSQTMGHDKMYWYQQDPGME
LHLIHYSYGVNSTEKGDLSSESTVSRIRTEHFPLTLESARPSHTSQYLC [symbol] [symbol]
FGEGSRLTVLEDLKNVFPPEVAVFEPSEAEISHTQKATLVCLATGFYPDHVELSWWVN
GKEVHSGVSTDPQPLKEQPALNDSRYCLSSRLRVSATFWQNPRNHFRCQVQFYGLSEND
EWTQDRAKPVTQIVSAEAWGRADCGFTSESYQQGVLSATILYEILLGKATLYAVLVSALVLM
AMVKRKDSRG
SEQ ID NO 032: TCR beta chain clone 11 (LMB1F3), variable domain, [symbol]
MSIGLLCCAALSLLWAGPVNAGVTQTPKFQVLKTGQSMTLQCAQDMNHEYMSWYRQDPG
MGLRLIHYSVGAGITDQGEVPNGYNVSRSTTEDFPLRLLSAAPSQTSVYFC [symbol] [symbol]
FGNGTRLTVTEDLNKVFPPEVAVFEPSEAEISHTQKATLVCLATGFFPDHVELSWW
VNGKEVHSGVSTDPQPLKEQPALNDSRYCLSSRLRVSATFWQNPRNHFRCQVQFYGLSE
NDEWTQDRAKPVTQIVSAEAWGRADCGFTSVSYQQGVLSATILYEILLGKATLYAVLVSALV
LMAMVKRKDF
SEQ ID NO 033: TCR beta chain clone 12 (MCA3D9), variable domain, [symbol]
MGFRLLCCVAFCLLGAGPVDSGVTQTPKHLITATGQRVTLRCSPRSGDLSVYWYQQSLDQ
GLQFLIQYYNGEERAKGNILERFSAQQFPDLHSELNLSSLELGDSALYFC [symbol] [symbol]
FGPGTRLTVLEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQFYGLSENDE
WTQDRAKPVTQIVSAEAWGRADCGFTSESYQQGVLSATILYEILLGKATLYAVLVSALVLMA
MVKRKDSRG
SEQ ID NO 034: TCR beta chain clone 13 (TC5A87), variable domain, [symbol]
MTIRLLCYMGFYFLGAGLMEADIYQTPRYLVIGTGKKITLECSQTMGHDKMYWYQQDPGME
LHLIHYSYGVNSTEKGDLSSESTVSRIRTEHFPLTLESARPSHTSQYLC [symbol]
FGEGSWLTVVEDLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFYGLSENDEWT
QDRAKPVTQIVSAEAWGRADCGFTSVSYQQGVLSATILYEILLGKATLYAVLVSALVLMAMV
KRKDF
SEQ ID NO 035: TCR beta chain clone 14 (SMC3), variable domain, [symbol]
MLLLLLLLGPGSGLGAVVSQHPSWVICKSGTSVKIECRSLDFQATTMFWYRQFPKQSLMLM
ATSNEGSKATYEQGVEKDKFLINHASLTLSTLTVTSAHPEDSSFYIC [symbol] [symbol]
FGPGTRLTVLEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQFYGLSENDE
WTQDRAKPVTQIVSAEAWGRADCGFTSESYQQGVLSATILYEILLGKATLYAVLVSALVLMA
MVKRKDSRG
SEQ ID NO 036: TCR beta chain clone 15 (TRA44), variable domain, [symbol]
MLSLLLLLLGLGSVFSAVISQKPSRDICQRGTSLTIQCQVDSQVTMMFWYRQQPGQSLTLIA
TANQGSEATYESGFVIDKFPISRPNLTFSTLTVSNMSPEDSSIYLC [symbol] FGPGTR
LTVTEDLKNVFPPEVAVFEPSEAEISHTQKATLVCLATGFYPDHVELSWWVNGKEVHSGVS
TDPQPLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKRKDSR
G
SEQ ID NO 037:
atgtggggagttttccttctttatgtttccatgaagatgggaggcactacaggacaaaacattgaccagcccactgagatgacagct
acggaaggtgccattgtccagatcaactgcacgtaccagacatctgggttcaacgggctgttctggtaccagcaacatgctggcg
aagcacccacatttctgtcttacaatgttctggatggtttggaggagaaaggtcgtttttcttcattccttagtcggtctaaagggtacag
ttacctccttttgaaggagctccagatgaaagactctgcctcttacctctgtgctgtgatggatagcagctataaattgatcttcgggag
tgggaccagactgctggtcaggcctgatatccagaaccctgaccctgccgtgtaccagctgagagactctaaatccagtgacaa
gtctgtctgcctattcaccgattttgattctcaaacaaatgtgtcacaaagtaaggattctgatgtgtatatcacagacaaaactgtgct
agacatgaggtctatggacttcaagagcaacagtgctgtggcctggagcaacaaatctgactttgcatgtgcaaacgccttcaac
aacagcattattccagaagacaccttcttccccagcccagaaagttcctgtgatgtcaagctggtcgagaaaagctttgaaacag
atacgaacctaaactttcaaaacctgtcagtgattgggttccgaatcctcctcctgaaagtggccgggtttaatctgctcatgacgct
gcggctgtggtccagc
SEQ ID NO 038:
atgctactcatcacatcaatgttggtcttatggatgcaattgtcacaggtgaatggacaacaggtaatgcaaattcctcagtaccagc
atgtacaagaaggagaggacttcaccacgtactgcaattcctcaactactttaagcaatatacagtggtataagcaaaggcctgg
tggacatcccgtttttttgatacagttagtgaagagtggagaagtgaagaagcagaaaagactgacatttcagtttggagaagcaa
aaaagaacagctccctgcacatcacagccacccagactacagatgtaggaacctacttctgtgcggctgctggtggtactagcta
tggaaagctgacatttggacaagggaccatcttgactgtccatccaaatatccagaaccctgaccctgccgtgtaccagctgaga
gactctaaatccagtgacaagtctgtctgcctattcaccgattttgattctcaaacaaatgtgtcacaaagtaaggattctgatgtgtat
atcacagacaaaactgtgctagacatgaggtctatggacttcaagagcaacagtgctgtggcctggagcaacaaatctgactttg
catgtgcaaacgccttcaacaacagcattattccagaagacaccttcttccccagcccagaaagttcctgtgatgtcaagctggtc
gagaaaagctttgaaacagatacgaacctaaactttcaaaacctgtcagtgattgggttccgaatcctcctcctgaaagtggccgg
gtttaatctgctcatgacgctgcggctgtggtccagc
SEQ ID NO 039:
atgaagacatttgctggattttcgttcctgtttttgtggctgcagctggactgtatgagtagaggagaggatgtggagcagagtcttttc
ctgagtgtccgagagggagacagctccgttataaactgcacttacacagacagctcctccacctacttatactggtataagcaag
aacctggagcaggtctccagttgctgacgtatattttttcaaatatggacatgaaacaagaccaaagactcactgttctattgaataa
aaaggataaacatctgtctctgcgcattgcagacacccagactggggactcagctatctacttctgtgcagagacctggaccgac
agaggctcaaccctggggaggctatactttggaagaggaactcagttgactgtctggcctgatatccagaaccctgaccctgccg
tgtaccagctgagagactctaaatccagtgacaagtctgtctgcctattcaccgattttgattctcaaacaaatgtgtcacaaagtaa
ggattctgatgtgtatatcacagacaaaactgtgctagacatgaggtctatggacttcaagagcaacagtgctgtggcctggagca
acaaatctgactttgcatgtgcaaacgccttcaacaacagcattattccagaagacaccttcttccccagcccagaaagttcctgtg
atgtcaagctggtcgagaaaagctttgaaacagatacgaacctaaactttcaaaacctgtcagtgattgggttccgaatcctcctcc
tgaaagtggccgggtttaatctgctcatgacgttgcggctgtggtccagc
SEQ ID NO 040:
atggccatgctcctgggggcatcagtgctgattctgtggcttcagacagactgggtaaacagtcaacagaagaatgatgaccagc
aagttaagcaaaattcaccatccctgagcgtccaggaaggaagaatttctattctgaactgtgactatactaacagcatgtttgatta
tttcctatggtacaaaaaataccctgctgaaggtcctacattcctgatatctataagttccattaaggataaaaatgaagatggaaga
ttcactgtcttcttaaacaaaagtgccaagcacctctctctgcacattgtgccctcccagcctggagactctgcagtgtacttctgtgca
gcaagtctttataaccagggaggaaagcttatcttcggacagggaacggagttatctgtgaaacccaatatccagaaccctgacc
ctgccgtgtaccagctgagagactctaaatccagtgacaagtctgtctgcctattcaccgattttgattctcaaacaaatgtgtcaca
aagtaaggattctgatgtgtatatcacagacaaaactgtgctagacatgaggtctatggacttcaagagcaacagtgctgtggcct
ggagcaacaaatctgactttgcatgtgcaaacgccttcaacaacagcattattccagaagacaccttcttccccagcccagaaag
ttcctgtgatgtcaagctggtcgagaaaagctttgaaacagatacgaacctaaactttcaaaacctgtcagtgattgggttccgaat
cctcctcctgaaagtggccgggtttaatctgctcatgacgctgcggctgtggtccagc
SEQ ID NO 041:
atggagaagaatcctttggcagccccattactaatcctctggtttcatcttgactgcgtgagcagcatactgaacgtggaacaaagt
cctcagtcactgcatgttcaggagggagacagcaccaatttcacctgcagcttcccttccagcaatttttatgccttacactggtaca
gatgggaaactgcaaaaagccccgaggccttgtttgtaatgactttaaatggggatgaaaagaagaaaggacgaataagtgcc
actcttaataccaaggagggttacagctatttgtacatcaaaggatcccagcctgaagactcagccacatacctctgtgcctcggg
ggattccgggtatgcactcaacttcggcaaaggcacctcgctgttggtcacaccccatatccagaaccctgaccctgccgtgtacc
agctgagagactctaaatccagtgacaagtctgtctgcctattcaccgattttgattctcaaacaaatgtgtcacaaagtaaggattct
gatgtgtatatcacagacaaaactgtgctagacatgaggtctatggacttcaagagcaacagtgctgtggcctggagcaacaaat
ctgactttgcatgtgcaaacgccttcaacaacagcattattccagaagacaccttcttccccagcccagaaagttcctgtgatgtca
agctggtcgagaaaagctttgaaacagatacgaacctaaactttcaaaacctgtcagtgattgggttccgaatcctcctcctgaaa
gtggccgggtttaatctgctcatgacgctgcggctgtggtccagc
SEQ ID NO 042:
atgaactattctccaggcttagtatctctgatactcttactgcttggaagaacccgtggaaattcagtgacccagatggaagggcca
gtgactctctcagaagaggccttcctgactataaactgcacgtacacagccacaggatacccttcccttttctggtatgtccaatatc
ctggagaaggtctacagctcctcctgaaagccacgaaggctgatgacaagggaagcaacaaaggttttgaagccacataccgt
aaagaaaccacttctttccacttggagaaaggctcagttcaagtgtcagactcagcggtgtacttctgtgctctgacaatatgggatt
atggaggaagccaaggaaatctcatctttggaaaaggcactaaactctctgttaaaccaaatatccagaaccctgaccctgccgt
gtaccagctgagagactctaaatccagtgacaagtctgtctgcctattcaccgattttgattctcaaacaaatgtgtcacaaagtaag
gattctgatgtgtatatcacagacaaaactgtgctagacatgaggtctatggacttcaagagcaacagtgctgtggcctggagcaa
caaatctgactttgcatgtgcaaacgccttcaacaacagcattattccagaagacaccttcttccccagcccagaaagttcctgtga
tgtcaagctggtcgagaaaagctttgaaacagatacgaacctaaactttcaaaacctgtcagtgattgggttccgaatcctcctcct
gaaagtggccgggtttaatctgctcatgacgctgcggctgtggtccagc
SEQ ID NO 043:
atggtcctgaaattctccgtgtccattctttggattcagttggcatgggtgagcacccagctgctggagcagagccctcagtttctaag
catccaagagggagaaaatctcactgtgtactgcaactcctcaagtgttttttccagcttacaatggtacagacaggagcctgggg
aaggtcctgtcctcctggtgacagtagttacgggtggagaagtgaagaagctgaagagactaacctttcagtttggtgatgcaaga
aaggacagttctctccacatcactgcagcccagcctggtgatacaggcctctacctctgtgcaggagaaaattccgggtatgcact
caacttcggcaaaggcacctcgctgttggtcacaccccatatccagaaccctgaccctgccgtgtaccagctgagagactctaaa
tccagtgacaagtctgtctgcctattcaccgattttgattctcaaacaaatgtgtcacaaagtaaggattctgatgtgtatatcacagac
aaaactgtgctagacatgaggtctatggacttcaagagcaacagtgctgtggcctggagcaacaaatctgactttgcatgtgcaa
acgccttcaacaacagcattattccagaagacaccttcttccccagcccagaaagttcctgtgatgtcaagctggtcgagaaaag
ctttgaaacagatacgaacctaaactttcaaaacctgtcagtgattgggttccgaatcctcctcctgaaagtggccgggtttaatctg
ctcatgacgctgcggctgtggtccagc
SEQ ID NO 044:
atgatgaaatccttgagagttttactggtgatcctgtggcttcagttaagctgggtttggagccaacagaaggaggtggagcaggat
cctggaccactcagtgttccagagggagccattgtttctctcaactgcacttacagcaacagtgcttttcaatacttcatgtggtacag
acagtattccagaaaaggccctgagttgctgatgtacacatactccagtggtaacaaagaagatggaaggtttacagcacaggt
cgataaatccagcaagtatatctccttgttcatcagagactcacagcccagtgattcagccacctacctctgtgcaatgagcctatc
aggaggaagctacatacctacatttggaagaggaaccagccttattgttcatccgtatatccagaaccctgaccctgccgtgtacc
agctgagagactctaaatccagtgacaagtctgtctgcctattcaccgattttgattctcaaacaaatgtgtcacaaagtaaggattct
gatgtgtatatcacagacaaaactgtgctagacatgaggtctatggacttcaagagcaacagtgctgtggcctggagcaacaaat
ctgactttgcatgtgcaaacgccttcaacaacagcattattccagaagacaccttcttccccagcccagaaagttcctgtgatgtca
agctggtcgagaaaagctttgaaacagatacgaacctaaactttcaaaacctgtcagtgattgggttccgaatcctcctcctgaaa
gtggccgggtttaatctgctcatgacgctgcggctgtggtccagc
SEQ ID NO 045:
atgctccttgaacatttattaataatcttgtggatgcagctgacatgggtcagtggtcaacagctgaatcagagtcctcaatctatgttt
atccaggaaggagaagatgtctccatgaactgcacttcttcaagcatatttaacacctggctatggtacaagcaggaccctgggg
aaggtcctgtcctcttgatagccttatataaggctggtgaattgacctcaaatggaagactgactgctcagtttggtataaccagaaa
ggacagcttcctgaatatctcagcatccatacctagtgatgtaggcatctacttctgtgctgggcagctaggaggggctggtggtact
agctatggaaagctgacatttggacaagggaccatcttgactgtccatccaaatatccagaaccctgaccctgccgtgtaccagct
gagagactctaaatccagtgacaagtctgtctgcctattcaccgattttgattctcaaacaaatgtgtcacaaagtaaggattctgat
gtgtatatcacagacaaaactgtgctagacatgaggtctatggacttcaagagcaacagtgctgtggcctggagcaacaaatctg
actttgcatgtgcaaacgccttcaacaacagcattattccagaagacaccttcttccccagcccagaaagttcctgtgatgtcaagc
tggtcgagaaaagctttgaaacagatacgaacctaaactttcaaaacctgtcagtgattgggttccgaatcctcctcctgaaagtgg
ccgggtttaatctgctcatgacgctgcggctgtggtccagc
SEQ ID NO 046:
atgacatccattcgagctgtatttatattcctgtggctgcagctggacttggtgaatggagagaatgtggagcagcatccttcaaccct
gagtgtccaggagggagacagcgctgttatcaagtgtacttattcagacagtgcctcaaactacttcccttggtataagcaagaact
tggaaaaggacctcagcttattatagacattcgttcaaatgtgggcgaaaagaaagaccaacgaattgctgttacattgaacaag
acagccaaacatttctccctgcacatcacagagacccaacctgaagactcggctgtctacttctgtgcagcaaactggagcccgc
aaggaaatgagaaattaacctttgggactggaacaagactcaccatcatacccaatatccagaaccctgaccctgccgtgtacc
agctgagagactctaaatccagtgacaagtctgtctgcctattcaccgattttgattctcaaacaaatgtgtcacaaagtaaggattct
gatgtgtatatcacagacaaaactgtgctagacatgaggtctatggacttcaagagcaacagtgctgtggcctggagcaacaaat
ctgactttgcatgtgcaaacgccttcaacaacagcattattccagaagacaccttcttccccagcccagaaagttcctgtgatgtca
agctggtcgagaaaagctttgaaacagatacgaacctaaactttcaaaacctgtcagtgattgggttccgaatcctcctcctgaaa
gtggccgggtttaatctgctcatgacgctgcggctgtggtccagc
SEQ ID NO 047:
atgtggggagttttccttctttatgtttccatgaagatgggaggcactacaggacaaaacattgaccagcccactgagatgacagct
acggaaggtgccattgtccagatcaactgcacgtaccagacatctgggttcaacgggctgttctggtaccagcaacatgctggcg
aagcacccacatttctgtcttacaatgttctggatggtttggaggagaaaggtcgtttttcttcattccttagtcggtctaaagggtacag
ttacctccttttgaaggagctccagatgaaagactctgcctcttacctctgtgcttccatggatagcaactatcagttaatctggggcgc
tgggaccaagctaattataaagccagatatccagaaccctgaccctgccgtgtaccagctgagagactctaaatccagtgacaa
gtctgtctgcctattcaccgattttgattctcaaacaaatgtgtcacaaagtaaggattctgatgtgtatatcacagacaaaactgtgct
agacatgaggtctatggacttcaagagcaacagtgctgtggcctggagcaacaaatctgactttgcatgtgcaaacgccttcaac
aacagcattattccagaagacaccttcttccccagcccagaaagttcctgtgatgtcaagctggtcgagaaaagctttgaaacag
atacgaacctaaactttcaaaacctgtcagtgattgggttccgaatcctcctcctgaaagtggccgggtttaatctgctcatgacgct
gcggctgtggtccagc
SEQ ID NO 048:
atgatatccttgagagttttactggtgatcctgtggcttcagttaagctgggtttggagccaacggaaggaggtggagcaggatcctg
gacccttcaatgttccagagggagccactgtcgctttcaactgtacttacagcaacagtgcttctcagtctttcttctggtacagacag
gattgcaggaaagaacctaagttgctgatgtccgtatactccagtggtaatgaagatggaaggtttacagcacagctcaatagag
ccagccagtatatttccctgctcatcagagactccaagctcagtgattcagccacctacctctgtgtggtgaacagattcacaaggg
atggaaacaaactggtctttggcgcaggaaccattctgagagtcaagtcctatatccagaaccctgaccctgccgtgtaccagctg
agagactctaaatccagtgacaagtctgtctgcctattcaccgattttgattctcaaacaaatgtgtcacaaagtaaggattctgatgt
gtatatcacagacaaaactgtgctagacatgaggtctatggacttcaagagcaacagtgctgtggcctggagcaacaaatctga
ctttgcatgtgcaaacgccttcaacaacagcattattccagaagacaccttcttccccagcccagaaagttcctgtgatgtcaagct
ggtcgagaaaagctttgaaacagatacgaacctaaactttcaaaacctgtcagtgattgggttccgaatcctcctcctgaaagtgg
ccgggtttaatctgctcatgacgctgcggctgtggtccagc
SEQ ID NO 049:
atgagcatcggcctcctgtgctgtgtggccttttctctcctgtgggcaagtccagtgaatgctggtgtcactcagaccccaaaattcca
ggtcctgaagacaggacagagcatgacactgcagtgtgcccaggatatgaaccataactccatgtactggtatcgacaagacc
caggcatgggactgaggctgatttattactcagcttctgagggtaccactgacaaaggagaagtccccaatggctacaatgtctcc
agattaaacaaacgggagttctcgctcaggctggagtcggctgctccctcccagacatctgtgtacttctgtgccagcagtgaggt
gacagggggatacaatgagcagttcttcgggccagggacacggctcaccgtgctagaggacctgaaaaacgtgttcccaccc
gaggtcgctgtgtttgagccatcagaagcagagatctcccacacccaaaaggccacactggtgtgcctggccacaggcttctac
cccgaccacgtggagctgagctggtgggtgaatgggaaggaggtgcacagtggggtcagcacagacccgcagcccctcaag
gagcagcccgccctcaatgactccagatactgcctgagcagccgcctgagggtctcggccaccttctggcagaacccccgcaa
ccacttccgctgtcaagtccagttctacgggctctcggagaatgacgagtggacccaggatagggccaaacctgtcacccagat
cgtcagcgccgaggcctggggtagagcagactgtggcttcacctccgagtcttaccagcaaggggtcctgtctgccaccatcctct
atgagatcttgctagggaaggccaccttgtatgccgtgctggtcagtgccctcgtgctgatggccatggtcaagagaaaggattcc
agaggc
SEQ ID NO 050:
atgctgagtcttctgctccttctcctgggactaggctctgtgttcagtgctgtcatctctcaaaagccaagcagggatatctgtcaacgt
ggaacctccctgacgatccagtgtcaagtcgatagccaagtcaccatgatgttctggtaccgtcagcaacctggacagagcctga
cactgatcgcaactgcaaatcagggctctgaggccacatatgagagtggatttgtcattgacaagtttcccatcagccgcccaaac
ctaacattctcaactctgactgtgagcaacatgagccctgaagacagcagcatatatctctgcagcgttggggggggcaagga
ccttacacagatacgcagtattttggcccaggcacccggctgacagtgctcgaggacctgaaaaacgtgttcccacccgaggtc
gctgtgtttgagccatcagaagcagagatctcccacacccaaaaggccacactggtgtgcctggccacaggcttctaccccgac
cacgtggagctgagctggtgggtgaatgggaaggaggtgcacagtggggtcagcacagacccgcagcccctcaaggagcag
cccgccctcaatgactccagatactgcctgagcagccgcctgagggtctcggccaccttctggcagaacccccgcaaccacttc
cgctgtcaagtccagttctacgggctctcggagaatgacgagtggacccaggatagggccaaacctgtcacccagatcgtcagc
gccgaggcctggggtagagcagactgtggcttcacctccgagtcttaccagcaaggggtcctgtctgccaccatcctctatgagat
cttgctagggaaggccaccttgtatgccgtgctggtcagtgccctcgtgctgatggccatggtcaagagaaaggattccagaggc
SEQ ID NO 051:
atgggaatcaggctcctgtgtcgtgtggccttttgtttcctggctgtaggcctcgtagatgtgaaagtaacccagagctcgagatatct
agtcaaaaggacgggagagaaagtttttctggaatgtgtccaggatatggaccatgaaaatatgttctggtatcgacaagaccca
ggtctggggctacggctgatctatttctcatatgatgttaaaatgaaagaaaaaggagatattcctgaggggtacagtgtctctaga
gagaagaaggagcgcttctccctgattctggagtccgccagcaccaaccagacatctatgtacctctgtgccagcagcttagggg
cgacaggggctaatgaaaaactgttttttggcagtggaacccagctctctgtcttggaggacctgaacaaggtgttcccacccgag
gtcgctgtgtttgagccatcagaagcagagatctcccacacccaaaaggccacactggtgtgcctggccacaggcttcttccctg
accacgtggagctgagctggtgggtgaatgggaaggaggtgcacagtggggtcagcacggacccgcagcccctcaaggagc
agcccgccctcaatgactccagatactgcctgagcagccgcctgagggtctcggccaccttctggcagaacccccgcaaccact
tccgctgtcaagtccagttctacgggctctcggagaatgacgagtggacccaggatagggccaaacccgtcacccagatcgtca
gcgccgaggcctggggtagagcagactgtggctttacctcggtgtcctaccagcaaggggtcctgtctgccaccatcctctatgag
atcctgctagggaaggccaccctgtatgctgtgctggtcagcgcccttgtgttgatggccatggtcaagagaaaggatttc
SEQ ID NO 052:
atgggctcctggaccctctgctgtgtgtccctttgcatcctggtagcaaagcacacagatgctggagttatccagtcaccccggcac
gaggtgacagagatgggacaagaagtgactctgagatgtaaaccaatttcaggacacgactaccttttctggtacagacagacc
atgatgcggggactggagttgctcatttactttaacaacaacgttccgatagatgattcagggatgcccgaggatcgattctcagct
aagatgcctaatgcatcattctccactctgaagatccagccctcagaacccagggactcagctgtgtacttctgtgccagcagcta
caggggcactgaagctttctttggacaaggcaccagactcacagttgtagaggacctgaacaaggtgttcccacccgaggtcgc
tgtgtttgagccatcagaagcagagatctcccacacccaaaaggccacactggtgtgcctggccacaggcttcttccctgaccac
gtggagctgagctggtgggtgaatgggaaggaggtgcacagtggggtcagcacggacccgcagcccctcaaggagcagccc
gccctcaatgactccagatactgcctgagcagccgcctgagggtctcggccaccttctggcagaacccccgcaaccacttccgct
gtcaagtccagttctacgggctctcggagaatgacgagtggacccaggatagggccaaacccgtcacccagatcgtcagcgcc
gaggcctggggtagagcagactgtggctttacctcggtgtcctaccagcaaggggtcctgtctgccaccatcctctatgagatcctg
ctagggaaggccaccctgtatgctgtgctggtcagcgcccttgtgttgatggccatggtcaagagaaaggatttc
SEQ ID NO 053:
atgggccctgggctcctctgctgggtgctgctttgtctcctgggagcaggcccagtggacgctggagtcacccaaagtcccacac
acctgatcaaaacgagaggacagcacgtgactctgagatgctctcctatctctgggcacaagagtgtgtcctggtaccaacaggt
cctgggtcaggggccccagtttatctttcagtattatgagaaagaagagagaggaagaggaaacttccctgatcgattctcagctc
gccagttccctaactatagctctgagctgaatgtgaacgccttgttgctgggggactcggccctgtatctctgtgccagcagctttgac
gttggtttgccacccctccactttgggaacgggaccaggctcactgtgacagaggacctgaacaaggtgttcccacccgaggtcg
ctgtgtttgagccatcagaagcagagatctcccacacccaaaaggccacactggtgtgcctggccacaggcttcttccctgacca
cgtggagctgagctggtgggtgaatgggaaggaggtgcacagtggggtcagcacggacccgcagcccctcaaggagcagcc
cgccctcaatgactccagatactgcctgagcagccgcctgagggtctcggccaccttctggcagaacccccgcaaccacttccg
ctgtcaagtccagttctacgggctctcggagaatgacgagtggacccaggatagggccaaacccgtcacccagatcgtcagcg
ccgaggcctggggtagagcagactgtggctttacctcggtgtcctaccagcaaggggtcctgtctgccaccatcctctatgagatc
ctgctagggaaggccaccctgtatgctgtgctggtcagcgcccttgtgttgatggccatggtcaagagaaaggatttc
SEQ ID NO 054:
atgggtcctgggcttctccactggatggccctttgtctccttggaacaggtcatggggatgccatggtcatccagaacccaagatac
caggttacccagtttggaaagccagtgaccctgagttgttctcagactttgaaccataacgtcatgtactggtaccagcagaagtca
agtcaggccccaaagctgctgttccactactatgacaaagattttaacaatgaagcagacacccctgataacttccaatccagga
ggccgaacacttctttctgctttcttgacatccgctcaccaggcctgggggacgcagccatgtacctgtgtgccaccagcagagag
tgggagacccagtacttcgggccaggcacgcggctcctggtgctcgaggacctgaaaaacgtgttcccacccgaggtcgctgtg
tttgagccatcagaagcagagatctcccacacccaaaaggccacactggtgtgcctggccacaggcttctaccccgaccacgtg
gagctgagctggtgggtgaatgggaaggaggtgcacagtggggtcagcacagacccgcagcccctcaaggagcagcccgc
cctcaatgactccagatactgcctgagcagccgcctgagggtctcggccaccttctggcagaacccccgcaaccacttccgctgt
caagtccagttctacgggctctcggagaatgacgagtggacccaggatagggccaaacctgtcacccagatcgtcagcgccga
ggcctggggtagagcagactgtggcttcacctccgagtcttaccagcaaggggtcctgtctgccaccatcctctatgagatcttgct
agggaaggccaccttgtatgccgtgctggtcagtgccctcgtgctgatggccatggtcaagagaaaggattccagaggc
SEQ ID NO 055:
atgactatcaggctcctctgctacatgggcttttattttctgggggcaggcctcatggaagctgacatctaccagaccccaagatacc
ttgttatagggacaggaaagaagatcactctggaatgttctcaaaccatgggccatgacaaaatgtactggtatcaacaagatcc
aggaatggaactacacctcatccactattcctatggagttaattccacagagaagggagatctttcctctgagtcaacagtctccag
aataaggacggagcattttcccctgaccctggagtctgccaggccctcacatacctctcagtacctctgtgccagcagccaacttta
ccgggacacctcgaacaccggggagctgttttttggagaaggctctaggctgaccgtactggaggacctgaaaaacgtgttccc
acccgaggtcgctgtgtttgagccatcagaagcagagatctcccacacccaaaaggccacactggtgtgcctggccacaggctt
ctaccccgaccacgtggagctgagctggtgggtgaatgggaaggaggtgcacagtggggtcagcacagacccgcagcccctc
aaggagcagcccgccctcaatgactccagatactgcctgagcagccgcctgagggtctcggccaccttctggcagaacccccg
caaccacttccgctgtcaagtccagttctacgggctctcggagaatgacgagtggacccaggatagggccaaacctgtcaccca
gatcgtcagcgccgaggcctggggtagagcagactgtggcttcacctccgagtcttaccagcaaggggtcctgtctgccaccatc
ctctatgagatcttgctagggaaggccaccttgtatgccgtgctggtcagtgccctcgtgctgatggccatggtcaagagaaaggat
tccagaggc
SEQ ID NO 056:
atgagcatcggcctcctgtgctgtgcagccttgtctctcctgtgggcaggtccagtgaatgctggtgtcactcagaccccaaaattcc
aggtcctgaagacaggacagagcatgacactgcagtgtgcccaggatatgaaccatgaatacatgtcctggtatcgacaagac
ccaggcatggggctgaggctgattcattactcagttggtgctggtatcactgaccaaggagaagtccccaatggctacaatgtctc
cagatcaaccacagaggatttcccgctcaggctgctgtcggctgctccctcccagacatctgtgtacttctgtgccagcggaatcag
cgggacagcgagctcctataattcacccctccactttgggaacgggaccaggctcactgtgacagaggacctgaacaaggtgtt
cccacccgaggtcgctgtgtttgagccatcagaagcagagatctcccacacccaaaaggccacactggtgtgcctggccacag
gcttcttccctgaccacgtggagctgagctggtgggtgaatgggaaggaggtgcacagtggggtcagcacggacccgcagccc
ctcaaggagcagcccgccctcaatgactccagatactgcctgagcagccgcctgagggtctcggccaccttctggcagaaccc
ccgcaaccacttccgctgtcaagtccagttctacgggctctcggagaatgacgagtggacccaggatagggccaaacccgtca
cccagatcgtcagcgccgaggcctggggtagagcagactgtggctttacctcggtgtcctaccagcaaggggtcctgtctgccac
catcctctatgagatcctgctagggaaggccaccctgtatgctgtgctggtcagcgcccttgtgttgatggccatggtcaagagaaa
ggatttc
SEQ ID NO 057:
atgggcttcaggctcctctgctgtgtggccttttgtctcctgggagcaggcccagtggattctggagtcacacaaaccccaaagcac
ctgatcacagcaactggacagcgagtgacgctgagatgctcccctaggtctggagacctctctgtgtactggtaccaacagagcc
tggaccagggcctccagttcctcattcagtattataatggagaagagagagcaaaaggaaacattcttgaacgattctccgcaca
acagttccctgacttgcactctgaactaaacctgagctctctggagctgggggactcagctttgtatttctgtgccagcagcgtcgga
gggggattggcagatacgcagtattttggcccaggcacccggctgacagtgctcgaggacctgaaaaacgtgttcccacccga
ggtcgctgtgtttgagccatcagaagcagagatctcccacacccaaaaggccacactggtgtgcctggccacaggcttctacccc
gaccacgtggagctgagctggtgggtgaatgggaaggaggtgcacagtggggtcagcacagacccgcagcccctcaaggag
cagcccgccctcaatgactccagatactgcctgagcagccgcctgagggtctcggccaccttctggcagaacccccgcaacca
cttccgctgtcaagtccagttctacgggctctcggagaatgacgagtggacccaggatagggccaaacctgtcacccagatcgtc
agcgccgaggcctggggtagagcagactgtggcttcacctccgagtcttaccagcaaggggtcctgtctgccaccatcctctatg
agatcttgctagggaaggccaccttgtatgccgtgctggtcagtgccctcgtgctgatggccatggtcaagagaaaggattccaga
ggc
SEQ ID NO 058:
atgactatcaggctcctctgctacatgggcttttattttctgggggcaggcctcatggaagctgacatctaccagaccccaagatacc
ttgttatagggacaggaaagaagatcactctggaatgttctcaaaccatgggccatgacaaaatgtactggtatcaacaagatcc
aggaatggaactacacctcatccactattcctatggagttaattccacagagaagggagatctttcctctgagtcaacagtctccag
aataaggacggagcattttcccctgaccctggagtctgccaggccctcacatacctctcagtacctctgtgccagcagtgaatatat
ccagtactctggaaacaccatatattttggagagggaagttggctcactgttgtagaggacctgaacaaggtgttcccacccgagg
tcgctgtgtttgagccatcagaagcagagatctcccacacccaaaaggccacactggtgtgcctggccacaggcttcttccctgac
cacgtggagctgagctggtgggtgaatgggaaggaggtgcacagtggggtcagcacggacccgcagcccctcaaggagcag
cccgccctcaatgactccagatactgcctgagcagccgcctgagggtctcggccaccttctggcagaacccccgcaaccacttc
cgctgtcaagtccagttctacgggctctcggagaatgacgagtggacccaggatagggccaaacccgtcacccagatcgtcag
cgccgaggcctggggtagagcagactgtggctttacctcggtgtcctaccagcaaggggtcctgtctgccaccatcctctatgaga
tcctgctagggaaggccaccctgtatgctgtgctggtcagcgcccttgtgttgatggccatggtcaagagaaaggatttc
SEQ ID NO 059:
atgctgctgcttctgctgcttctggggccaggctccgggcttggtgctgtcgtctctcaacatccgagctgggttatctgtaagagtgg
aacctctgtgaagatcgagtgccgttccctggactttcaggccacaactatgttttggtatcgtcagttcccgaaacagagtctcatgc
tgatggcaacttccaatgagggctccaaggccacatacgagcaaggcgtcgagaaggacaagtttctcatcaaccatgcaagc
ctgaccttgtccactctgacagtgaccagtgcccatcctgaagacagcagcttctacatctgcagtgcgaaggtgactagcgggc
aacaccaagggaccacagatacgcagtattttggcccaggcacccggctgacagtgctcgaggacctgaaaaacgtgttccca
cccgaggtcgctgtgtttgagccatcagaagcagagatctcccacacccaaaaggccacactggtgtgcctggccacaggcttc
taccccgaccacgtggagctgagctggtgggtgaatgggaaggaggtgcacagtggggtcagcacagacccgcagcccctc
aaggagcagcccgccctcaatgactccagatactgcctgagcagccgcctgagggtctcggccaccttctggcagaacccccg
caaccacttccgctgtcaagtccagttctacgggctctcggagaatgacgagtggacccaggatagggccaaacctgtcaccca
gatcgtcagcgccgaggcctggggtagagcagactgtggcttcacctccgagtcttaccagcaaggggtcctgtctgccaccatc
ctctatgagatcttgctagggaaggccaccttgtatgccgtgctggtcagtgccctcgtgctgatggccatggtcaagagaaaggat
tccagaggc
SEQ ID NO 060:
atgctgagtcttctgctccttctcctgggactaggctctgtgttcagtgctgtcatctctcaaaagccaagcagggatatctgtcaacgt
ggaacctccctgacgatccagtgtcaagtcgatagccaagtcaccatgatgttctggtaccgtcagcaacctggacagagcctga
cactgatcgcaactgcaaatcagggctctgaggccacatatgagagtggatttgtcattgacaagtttcccatcagccgcccaaac
ctaacattctcaactctgactgtgagcaacatgagccctgaagacagcagcatatatctctgcagcgttgaaggcaggggttacg
agcagtacttcgggccgggcaccaggctcacggtcacagaggacctgaaaaacgtgttcccacccgaggtcgctgtgtttgagc
catcagaagcagagatctcccacacccaaaaggccacactggtgtgcctggccacaggcttctaccccgaccacgtggagctg
agctggtgggtgaatgggaaggaggtgcacagtggggtcagcacagacccgcagcccctcaaggagcagcccgccctcaat
gactccagatactgcctgagcagccgcctgagggtctcggccaccttctggcagaacccccgcaaccacttccgctgtcaagtcc
agttctacgggctctcggagaatgacgagtggacccaggatagggccaaacctgtcacccagatcgtcagcgccgaggcctgg
ggtagagcagactgtggcttcacctccgagtcttaccagcaaggggtcctgtctgccaccatcctctatgagatcttgctagggaag
gccaccttgtatgccgtgctggtcagtgccctcgtgctgatggccatggtcaagagaaaggattccagaggc
SEQ ID NO 061: TCR gamma chain clone (1) (MGDA1G5), variable domain, [symbol]
MQWALAVLLAFLSPASQKSSNLEGRTKSVIRQTGSSAEITCDLAEGSTGYIHWYLHQEGKA
PQRLLYYDSYTSSVVLESGISPGKYDTYGSTRKNLRMILRNLIENDSGVYYC [symbol] [symbol]
FGPGTKLIITDKQLDADVSPKPTIFLPSIAETKLQKAGTYLCLLEKFFPDVIKIHWQEKKS
NTILGSQEGNTMKTNDTYMKFSWLTVPEKSLDKEHRCIVRHENNKNGVDQEIIFPPIKTDVIT
MDPKDNCSKDANDTLLLQLTNTSAYYMYLLLLLKSVVYFAIITCCLLRRTAFCCNGEKS
SEQ ID NO 062: TCR delta chain clone (1) (MGDA1G5), variable domain, [symbol]
MLFSSLLCVFVAFSYSGSSVAQKVTQAQSSVSMPVRKAVTLNCLYETSWWSYYIFWYKQLP
SKEMIFLIRQGSDEQNAKSGRYSVNFKKAVKSVALTISALQLEDSAKYFC [symbol] [symbol]
FGKGTRVTVEPRSQPHTKPSVFVMKNGTNVACLVKEFYPKDIRINLVSSKKITEFDP
AIVISPSGKYNAVKLGKYEDSNSVTCSVQHDNKTVHSTDFEVKTDSTDHVKPKETENTKQPS
KSCHKPKAIVHTEKVNMMSLTVLGLRMLFAKTVAVNFLLTAKLFFL
SEQ ID NO 063:
atgcagtgggccctagcggtgcttctagctttcctgtctcctgccagtcagaaatcttccaacttggaagggagaacgaagtcagtc
atcaggcagactgggtcatctgctgaaatcacttgtgatcttgctgaaggaagtaccggctacatccactggtacctacaccagga
ggggaaggccccacagcgtcttctgtactatgactcctacacctccagcgttgtgttggaatcaggaatcagcccagggaagtat
gatacttatggaagcacaaggaagaacttgagaatgatactgcgaaatcttattgaaaatgactctggagtctattactgtgccacc
tgggaaactcaagagttgggcaaaaaaatcaaggtatttggtcccggaacaaagcttatcattacagataaacaacttgatgcag
atgtttcccccaagcccactatttttcttccttcaattgctgaaacaaagctccagaaggctggaacatacctttgtcttcttgagaaattt
ttccctgatgttattaagatacattggcaagaaaagaagagcaacacgattctgggatcccaggaggggaacaccatgaagact
aacgacacatacatgaaatttagctggttaacggtgccagaaaagtcactggacaaagaacacagatgtatcgtcagacatga
gaataataaaaacggagttgatcaagaaattatctttcctccaataaagacagatgtcatcacaatggatcccaaagacaattgtt
caaaagatgcaaatgatacactactgctgcagctcacaaacacctctgcatattacatgtacctcctcctgctcctcaagagtgtgg
tctattttgccatcatcacctgctgtctgcttagaagaacggctttctgctgcaatggagagaaatca
SEQ ID NO 064:
atgctgttctccagcctgctgtgtgtatttgtggccttcagctactctggatcaagtgtggcccagaaggttactcaagcccagtcatca
gtatccatgccagtgaggaaagcagtcaccctgaactgcctgtatgaaacaagttggtggtcatattatattttttggtacaagcaac
ttcccagcaaagagatgattttccttattcgccagggttctgatgaacagaatgcaaaaagtggtcgctattctgtcaacttcaagaa
agcagtgaaatccgtcgccttaaccatttcagccttacagctagaagattcagcaaagtacttttgtgctcttggggtccaagccctc
ctacccatactgggggataccaccgataaactcatctttggaaaaggaacccgtgtgactgtggaaccaagaagtcagcctcat
accaaaccatccgtttttgtcatgaaaaatggaacaaatgtcgcttgtctggtgaaggaattctaccccaaggatataagaataaat
ctcgtgtcatccaagaagataacagagtttgatcctgctattgtcatctctcccagtgggaagtacaatgctgtcaagcttggtaaata
tgaagattcaaattcagtgacatgttcagttcaacacgacaataaaactgtgcactccactgactttgaagtgaagacagattctac
agatcacgtaaaaccaaaggaaactgaaaacacaaagcaaccttcaaagagctgccataaacccaaagccatagttcatac
cgagaaggtgaacatgatgtccctcacagtgcttgggctacgaatgctgtttgcaaagactgttgccgtcaattttctcttgactgcca
agttatttttcttg

We claim:

1. A T cell receptor (TCR) heterodimer comprising a TCR alpha chain (TCRα) polypeptide, and a TCR beta chain (TCRβ) polypeptide,
wherein said TCRα polypeptide and said TCRβ polypeptide are characterized respectively by a pair of a TCRα complementarity determining region 3 (CDR3) and a TCRβ CDR3 selected from SEQ ID NO 065 and SEQ ID NO 080, or SEQ ID NO 066 and SEQ ID NO 081, or SEQ ID NO 067 and SEQ ID NO 082, or SEQ ID NO 068 and SEQ ID NO 083, or SEQ ID NO 069 and SEQ ID NO 084, or SEQ ID NO 070 and 85, or SEQ ID NO 071 and SEQ ID NO 086, or SEQ ID NO 072 and SEQ ID NO 087, or SEQ ID NO 073 and SEQ ID NO 088, or SEQ ID NO 074 and SEQ ID NO 089, or SEQ ID NO 075 and SEQ ID NO 090, or SEQ ID NO 076 and SEQ ID NO 091, or SEQ ID NO 077 and SEQ ID NO 092, or SEQ ID NO 078 and SEQ ID NO 093, or SEQ ID NO 079 and SEQ ID NO 094.

2. The TCR heterodimer according to claim 1, wherein said TCRα polypeptide, and said TCRβ polypeptide are characterized by a pair of a TCRα variable domain and a TCRβ variable domain identical to those comprised within the sequences SEQ ID NO 001 and SEQ ID NO 002; or, SEQ ID NO 003 and SEQ ID NO 004; or SEQ ID NO 005 and SEQ ID NO 006, SEQ ID NO 013 and SEQ ID NO 025; or SEQ ID NO 014 and SEQ ID NO 026; or SEQ ID NO 015 and SEQ ID NO 027; or SEQ ID NO 016 and SEQ ID NO 028; or SEQ ID NO 017 and SEQ ID NO 029; or SEQ ID NO 018 and SEQ ID NO 030; or SEQ ID NO 019 and SEQ ID NO 031; or SEQ ID NO 20 and SEQ ID NO 032; or SEQ ID NO 021 and SEQ ID NO 033; or SEQ ID NO 022 and SEQ ID NO 034; or SEQ ID NO 023 and SEQ ID NO 035; or SEQ ID NO 024 and SEQ ID NO 036.

3. The TCR heterodimer according to claim 1, wherein
the TCRα polypeptide is at least (≥)85%, ≥90%, ≥95%, or ≥98 similar to SEQ ID NO 001 and has a TCRα CDR3 identical to SEQ ID NO 065, and the TCRβ polypeptide is ≥85%, ≥90%, ≥95%, or ≥98 similar to SEQ ID NO 002 and has a TCRβ CDR3 identical to SEQ ID NO 080; or
the TCRα polypeptide is ≥85%, ≥90%, ≥95%, or ≥98 similar to SEQ ID NO 003 and has a TCRα CDR3 identical to SEQ ID NO 066, and the TCRβ polypeptide is ≥85%, ≥90%, ≥95%, or ≥98 similar to SEQ ID NO 004 and has a TCRβ CDR3 identical to SEQ ID NO 081; or
the TCRα polypeptide is ≥85%, ≥90%, ≥95%, or ≥98 similar to SEQ ID NO 005 and has TCRα CDR3 identical to SEQ ID NO 067, and the TCRβ polypeptide is ≥85%, ≥90%, ≥95%, or ≥98 similar to SEQ ID NO 006 and has a TCRβ CDR3 identical to SEQ ID NO 082; or
the TCRα polypeptide is ≥85%, ≥90%, ≥95%, or ≥98 similar to SEQ ID NO 013 and has a TCRα CDR3 identical to SEQ ID NO 068, and the TCRβ polypeptide is ≥85%, ≥90%, ≥95%, or ≥98 similar to SEQ ID NO 025 and has a TCRβ CDR3 identical to SEQ ID NO 083; or
the TCRα polypeptide is ≥85%, ≥90%, ≥95%, or ≥98 similar to SEQ ID NO 014 and has a TCRα CDR3 identical to SEQ ID NO 069, and the TCRβ polypeptide is ≥85%, ≥90%, ≥95%, or ≥98 similar to SEQ ID NO 026 and has a TCRβ CDR3 identical to SEQ ID NO 084; or
the TCRα polypeptide is ≥85%, ≥90%, ≥95%, or ≥98 similar to SEQ ID NO 015 and has a TCRα CDR3 identical to SEQ ID NO 070, and the TCRβ polypeptide is ≥85%, ≥90%, ≥95%, or ≥98 similar to SEQ ID NO 027 and has a TCRβ CDR3 identical to SEQ ID NO 085; or
the TCRα polypeptide is ≥85%, ≥90%, ≥95%, or ≥98 similar to SEQ ID NO 016 and has a TCRα CDR3 identical to SEQ ID NO 071, and the TCRβ polypeptide is ≥85%, ≥90%, ≥95%, or ≥98 similar to SEQ ID NO 028 and has a TCRβ CDR3 identical to SEQ ID NO 086; or
the TCRα polypeptide is ≥85%, ≥90%, ≥95%, or ≥98 similar to SEQ ID NO 017 and has a TCRα CDR3 identical to SEQ ID NO 072, and the TCRβ polypeptide is ≥85%, ≥90%, ≥95%, or ≥98 similar to SEQ ID NO 029 and has a TCRβ CDR3 identical to SEQ ID NO 087; or
the TCRα polypeptide is ≥85%, ≥90%, ≥95%, or ≥98 similar to SEQ ID NO 017 and has a TCRα CDR3 identical to SEQ ID NO 072, and the TCRβ polypeptide is ≥85%, ≥90%, ≥95%, or ≥98 similar to SEQ ID NO 029 and has a TCRβ CDR3 identical to SEQ ID NO 087; or
the TCRα polypeptide is ≥85%, ≥90%, ≥95%, or ≥98 similar to SEQ ID NO 018 and has a TCRα CDR3 identical to SEQ ID NO 073, and the TCRβ polypeptide is ≥85%, ≥90%, ≥95%, or ≥98 similar to SEQ ID NO 030 and has a TCRβ CDR3 identical to SEQ ID NO 088; or
the TCRα polypeptide is ≥85%, ≥90%, ≥95%, or ≥98 similar to SEQ ID NO 019 and has a TCRα CDR3 identical to SEQ ID NO 074, and the TCRβ polypeptide is ≥85%, ≥90%, ≥95%, or ≥98 similar to SEQ ID NO 031 and has a TCRβ CDR3 identical to SEQ ID NO 089; or
the TCRα polypeptide is ≥85%, ≥90%, ≥95%, or ≥98 similar to SEQ ID NO 020 and has a TCRα CDR3 identical to SEQ ID NO 075, and the TCRβ polypeptide is ≥85%, ≥90%, ≥95%, or ≥98 similar to SEQ ID NO 032 and has a TCRβ CDR3 identical to SEQ ID NO 090; or
the TCRα polypeptide is ≥85%, ≥90%, ≥95%, or ≥98 similar to SEQ ID NO 021 and has a TCRα CDR3 identical to SEQ ID NO 076, and the TCRβ polypeptide is ≥85%, ≥90%, ≥95%, or ≥98 similar to SEQ ID NO 033 and has a TCRβ CDR3 identical to SEQ ID NO 091; or
the TCRα polypeptide is ≥85%, ≥90%, ≥95%, or ≥98 similar to SEQ ID NO 022 and has a TCRα CDR3 identical to SEQ ID NO 077, and the TCRβ polypeptide is ≥85%, ≥90%, ≥95%, or ≥98 similar to SEQ ID NO 034 and has a TCRβ CDR3 identical to SEQ ID NO 092; or
the TCRα polypeptide is ≥85%, ≥90%, ≥95%, or ≥98 similar to SEQ ID NO 022 and has a TCRα CDR3 identical to SEQ ID NO 077, and the TCRβ polypeptide is ≥85%, ≥90%, ≥95%, or ≥98 similar to SEQ ID NO 034 and has a TCRβ CDR3 identical to SEQ ID NO 092; or
the TCRα polypeptide is ≥85%, ≥90%, ≥95%, or ≥98 similar to SEQ ID NO 023 and has a TCRα CDR3 identical to SEQ ID NO 078, and the TCRβ polypeptide is ≥85%, ≥90%, ≥95%, or ≥98 similar to SEQ ID NO 035 and has a TCRβ CDR3 identical to SEQ ID NO 093; or
the TCRα polypeptide is ≥85%, ≥90%, ≥95%, or ≥98 similar to SEQ ID NO 024 and has a TCRα CDR3 identical to SEQ ID NO 079, and the TCRβ polypeptide is ≥85%, ≥90%, ≥95%, or ≥98 similar to SEQ ID NO 036 and has a TCRβ CDR3 identical to SEQ ID NO 094.

4. The TCR heterodimer according to claim 1, wherein the TCRα polypeptide, and the TCRα polypeptide amino acid sequence respectively are identical to SEQ ID NO 001 and SEQ ID NO 002; or, SEQ ID NO 003 and SEQ ID NO 004; or SEQ ID NO 005 and SEQ ID NO 006, SEQ ID NO 013 and SEQ ID NO 025; or SEQ ID NO 014 and SEQ ID NO 026; or SEQ ID NO 015 and SEQ ID NO 027; or SEQ ID NO 016 and SEQ ID NO 028; or SEQ ID NO 017 and SEQ ID NO 029; or SEQ ID NO 018 and SEQ ID NO 030; or SEQ ID NO 019 and SEQ ID NO 031; or SEQ ID NO 20 and SEQ ID NO 032; or SEQ ID NO 021 and SEQ ID NO 033; or SEQ ID NO 022 and SEQ ID NO 034; or SEQ ID NO 023 and SEQ ID NO 035; or SEQ ID NO 024 and SEQ ID NO 036.

5. The TCR heterodimer according to claim 1, that binds to an MR1 molecule presenting a tumour-associated antigen.

6. A soluble TCR heterodimer comprising the TCR heterodimer of claim 1.

7. A multimerized TCR heterodimer comprising the TCR heterodimer of claim 1.

8. An expression vector comprising a nucleic acid molecule encoding the TCR heterodimer of claim 1.

9. The expression vector of claim 5, comprising the nucleic acid sequences SEQ ID NO 007 and SEQ ID NO 008, or SEQ ID NO 009 and SEQ ID NO 010, or SEQ ID NO 011 and SEQ ID NO 012, or SEQ ID NO 037 and SEQ ID NO 049, or SEQ ID NO 038 and SEQ ID NO 050, or SEQ ID NO 038 and SEQ ID NO 050, or SEQ ID NO 039 and SEQ ID NO 051, or SEQ ID NO 040 and SEQ ID NO 052, or SEQ ID NO 041 and SEQ ID NO 053, or SEQ ID NO 042 and SEQ ID NO 054, or SEQ ID NO 043 and SEQ ID NO 055, or SEQ ID NO 044 and SEQ ID NO 056, or SEQ ID NO 045 and SEQ ID NO 057, or SEQ ID NO 046 and SEQ ID NO 058, or SEQ ID NO 047 and SEQ ID NO 059, or SEQ ID NO 048 and SEQ ID NO 060.

10. The expression vector of claim 8, wherein the expression vector is a lentivirus.

11. A recombinant cell comprising the TCR heterodimer of claim 1.

12. A preparation of recombinant T cells, wherein each T cell expresses as a transgene a TCR heterodimer according to claim 1.

13. A method for treatment of cancer in a patient, comprising administering to the patient a preparation of MR1-specific T cells, wherein each cell expresses as a transgene a TCR heterodimer according to claim 1.

14. The method according to claim 13, wherein the preparation of MR1-specific T cells is obtained from the same patient in an autologous adoptive T cell therapy.

15. The method according to claim 13, wherein the preparation of MR1-specific T cells is obtained from a donor in an allogeneic adoptive T cell therapy.

16. The method according to claim 13, wherein the preparation of MR1-specific T cells is obtained from an HLA-matched donor in an allogeneic adoptive T cell therapy.

17. The method according to claim 13, wherein the cancer is leukemia, melanoma, colon carcinoma, colon adenocarcinoma, breast cancer, hepatocellular carcinoma or lung cancer.

18. A recombinant cell comprising the expression vector of claim 7.


SEQUENCE LISTING (102 sequences)

SEQ ID NO: 1 - protein, 280 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAQIYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 309 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFSSGKQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 3 - protein, 269 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 313 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDVGTGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 5 - protein, 277 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEEPSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 313 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSRLLAGGQNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - DNA, 840 bp, Homo sapiens
  source 1..840: mol_type = genomic DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcccagatttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 8 - DNA, 927 bp, Homo sapiens
  source 1..927: mol_type = genomic DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttttctagcggaaagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 9 - DNA, 807 bp, Homo sapiens
  source 1..807: mol_type = genomic DNA
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtcggtatactccagtggtaatgaagatggaaggtttacagcacagctcaatagagccagccagtatatttccctgctcatcagagactccaagctcagtgattcagccacctacctctgtgtggtgaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 10 - DNA, 939 bp, Homo sapiens
  source 1..939: mol_type = genomic DNA
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatgtcgggacaggggacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 11 - DNA, 831 bp, Homo sapiens
  source 1..831: mol_type = genomic DNA
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggaacctagcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 12 - DNA, 939 bp, Homo sapiens
  source 1..939: mol_type = genomic DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtcgactactagcgggggggcagaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 13 - protein, 267 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 271 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAAAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 277 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAETWTDRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 280 aa, Homo sapiens
MAMLLGASVLILWLQTDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 274 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 276 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALTIWDYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 269 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGENSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 275 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSLSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 275 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQLGGAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 274 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAANWSPQGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 267 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 274 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNRFTRDGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 312 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEVTGGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 26 - protein, 311 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGAGQGPYTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 27 - protein, 311 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLGATGANEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 28 - protein, 308 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYRGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 309 aa, Homo sapiens
MGPGLLCWVLLCLLGAGPVDAGVTQSPTHLIKTRGQHVTLRCSPISGHKSVSWYQQVLGQGPQFIFQYYEKEERGRGNFPDRFSARQFPNYSSELNVNALLLGDSALYLCASSFDVGLPPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 30 - protein, 309 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREWETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 315 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSQLYRDTSNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 32 - protein, 313 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASGISGTASSYNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 312 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGGGLADTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 34 - protein, 311 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEYIQYSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 314 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAKVTSGQHQGTTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - protein, 307 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEGRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - DNA, 801 bp, Homo sapiens
  source 1..801: mol_type = genomic DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgatggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 38 - DNA, 813 bp, Homo sapiens
  source 1..813: mol_type = genomic DNA
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcggctgctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 39 - DNA, 831 bp, Homo sapiens
  source 1..831: mol_type = genomic DNA
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagacctggaccgacagaggctcaaccctggggaggctatactttggaagaggaactcagttgactgtctggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgttgcggctgtggtccagc

SEQ ID NO: 40 - DNA, 840 bp, Homo sapiens
  source 1..840: mol_type = genomic DNA
atggccatgctcctgggggcatcagtgctgattctgtggcttcagacagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagtctttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 41 - DNA, 822 bp, Homo sapiens
  source 1..822: mol_type = genomic DNA
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctcgggggattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 42 - DNA, 828 bp, Homo sapiens
  source 1..828: mol_type = genomic DNA
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgacaatatgggattatggaggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 43 - DNA, 807 bp, Homo sapiens
  source 1..807: mol_type = genomic DNA
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagaaaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 44 - DNA, 825 bp, Homo sapiens
  source 1..825: mol_type = genomic DNA
atgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcctatcaggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 45 - DNA, 825 bp, Homo sapiens
  source 1..825: mol_type = genomic DNA
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcagctaggaggggctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 46 - DNA, 822 bp, Homo sapiens
  source 1..822: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaaactggagcccgcaaggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 47 - DNA, 801 bp, Homo sapiens
  source 1..801: mol_type = genomic DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgcttccatggatagcaactatcagttaatctggggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 48 - DNA, 822 bp, Homo sapiens
  source 1..822: mol_type = genomic DNA
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacagattcacaagggatggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 49 - DNA, 936 bp, Homo sapiens
  source 1..936: mol_type = genomic DNA
atgagcatcggcctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaggtgacagggggatacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 50 - DNA, 933 bp, Homo sapiens
  source 1..933: mol_type = genomic DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttggggcggggcaaggaccttacacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 51 - DNA, 933 bp, Homo sapiens
  source 1..933: mol_type = genomic DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagcttaggggcgacaggggctaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 52 - DNA, 924 bp, Homo sapiens
  source 1..924: mol_type = genomic DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagctacaggggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 53 - DNA, 927 bp, Homo sapiens
  source 1..927: mol_type = genomic DNA
atgggccctgggctcctctgctgggtgctgctttgtctcctgggagcaggcccagtggacgctggagtcacccaaagtcccacacacctgatcaaaacgagaggacagcacgtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagctttgacgttggtttgccacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 54 - DNA, 927 bp, Homo sapiens
  source 1..927: mol_type = genomic DNA
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagagagtgggagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 55 - DNA, 945 bp, Homo sapiens
  source 1..945: mol_type = genomic DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagccaactttaccgggacacctcgaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 56 - DNA, 939 bp, Homo sapiens
  source 1..939: mol_type = genomic DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcggaatcagcgggacagcgagctcctataattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 57 - DNA, 936 bp, Homo sapiens
  source 1..936: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtcggagggggattggcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 58 - DNA, 933 bp, Homo sapiens
  source 1..933: mol_type = genomic DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtgaatatatccagtactctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 59 - DNA, 942 bp, Homo sapiens
  source 1..942: mol_type = genomic DNA
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcgaaggtgactagcgggcaacaccaagggaccacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 60 - DNA, 921 bp, Homo sapiens
  source 1..921: mol_type = genomic DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaggcaggggttacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 61 - protein, 310 aa, Homo sapiens
MQWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWETQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 62 - protein, 295 aa, Homo sapiens
MLFSSLLCVFVAFSYSGSSVAQKVTQAQSSVSMPVRKAVTLNCLYETSWWSYYIFWYKQLPSKEMIFLIRQGSDEQNAKSGRYSVNFKKAVKSVALTISALQLEDSAKYFCALGVQALLPILGDTTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 63 - DNA, 930 bp, Homo sapiens
  source 1..930: mol_type = genomic DNA
atgcagtgggccctagcggtgcttctagctttcctgtctcctgccagtcagaaatcttccaacttggaagggagaacgaagtcagtcatcaggcagactgggtcatctgctgaaatcacttgtgatcttgctgaaggaagtaccggctacatccactggtacctacaccaggaggggaaggccccacagcgtcttctgtactatgactcctacacctccagcgttgtgttggaatcaggaatcagcccagggaagtatgatacttatggaagcacaaggaagaacttgagaatgatactgcgaaatcttattgaaaatgactctggagtctattactgtgccacctgggaaactcaagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaatca

SEQ ID NO: 64 - DNA, 885 bp, Homo sapiens
  source 1..885: mol_type = genomic DNA
atgctgttctccagcctgctgtgtgtatttgtggccttcagctactctggatcaagtgtggcccagaaggttactcaagcccagtcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagtgaaatccgtcgccttaaccatttcagccttacagctagaagattcagcaaagtacttttgtgctcttggggtccaagccctcctacccatactgggggataccaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 65 - protein, 17 aa, Homo sapiens
CAAQIYNQGGKLIFGQG

SEQ ID NO: 66 - protein, 13 aa, Homo sapiens
CVVTGNQFYFGTG

SEQ ID NO: 67 - protein, 18 aa, Homo sapiens
CALSEEPSNTGKLIFGQG

SEQ ID NO: 68 - protein, 15 aa, Homo sapiens
CAVMDSSYKLIFGSG

SEQ ID NO: 69 - protein, 17 aa, Homo sapiens
CAAAGGTSYGKLTFGQG

SEQ ID NO: 70 - protein, 20 aa, Homo sapiens
CAETWTDRGSTLGRLYFGRG

SEQ ID NO: 71 - protein, 17 aa, Homo sapiens
CAASLYNQGGKLIFGQG

SEQ ID NO: 72 - protein, 15 aa, Homo sapiens
CASGDSGYALNFGKG

SEQ ID NO: 73 - protein, 20 aa, Homo sapiens
CALTIWDYGGSQGNLIFGKG

SEQ ID NO: 74 - protein, 15 aa, Homo sapiens
CAGENSGYALNFGKG

SEQ ID NO: 75 - protein, 17 aa, Homo sapiens
CAMSLSGGSYIPTFGRG

SEQ ID NO: 76 - protein, 21 aa, Homo sapiens
CAGQLGGAGGTSYGKLTFGQG

SEQ ID NO: 77 - protein, 18 aa, Homo sapiens
CAANWSPQGNEKLTFGTG

SEQ ID NO: 78 - protein, 15 aa, Homo sapiens
CASMDSNYQLIWGAG

SEQ ID NO: 79 - protein, 18 aa, Homo sapiens
CVVNRFTRDGNKLVFGAG

SEQ ID NO: 80 - protein, 15 aa, Homo sapiens
CASSFSSGKQYFGPG

SEQ ID NO: 81 - protein, 19 aa, Homo sapiens
CATSDVGTGDTGELFFGEG

SEQ ID NO: 82 - protein, 19 aa, Homo sapiens
CASSRLLAGGQNEQFFGPG

SEQ ID NO: 83 - protein, 18 aa, Homo sapiens
CASSEVTGGYNEQFFGPG

SEQ ID NO: 84 - protein, 19 aa, Homo sapiens
CSVGAGQGPYTDTQYFGPG

SEQ ID NO: 85 - protein, 19 aa, Homo sapiens
CASSLGATGANEKLFFGSG

SEQ ID NO: 86 - protein, 15 aa, Homo sapiens
CASSYRGTEAFFGQG

SEQ ID NO: 87 - protein, 17 aa, Homo sapiens
CASSFDVGLPPLHFGNG

SEQ ID NO: 88 - protein, 15 aa, Homo sapiens
CATSREWETQYFGPG

SEQ ID NO: 89 - protein, 21 aa, Homo sapiens
CASSQLYRDTSNTGELFFGEG

SEQ ID NO: 90 - protein, 21 aa, Homo sapiens
CASGISGTASSYNSPLHFGNG

SEQ ID NO: 91 - protein, 18 aa, Homo sapiens
CASSVGGGLADTQYFGPG

SEQ ID NO: 92 - protein, 19 aa, Homo sapiens
CASSEYIQYSGNTIYFGEG

SEQ ID NO: 93 - protein, 22 aa, Homo sapiens
CSAKVTSGQHQGTTDTQYFGPG

SEQ ID NO: 94 - protein, 15 aa, Homo sapiens
CSVEGRGYEQYFGPG

SEQ ID NO: 95 - protein, 19 aa, Homo sapiens
CATWETQELGKKIKVFGPG

SEQ ID NO: 96 - protein, 24 aa, Homo sapiens
CALGVQALLPILGDTTDKLIFGKG

SEQ ID NO: 97 - DNA, 22 bp, synthetic construct
  misc_feature 1..22: note = Primer
  source 1..22: mol_type = other DNA
ctcggcaggccgagccacgggc

SEQ ID NO: 98 - DNA, 22 bp, synthetic construct
  misc_feature 1..22: note = Primer
  source 1..22: mol_type = other DNA
gcccgtggctcggcctgccgag

SEQ ID NO: 99 - DNA, 27 bp, synthetic construct
  misc_feature 1..27: note = Primer
  source 1..27: mol_type = other DNA
ctcgagatgtctcgctccgtggcctta

SEQ ID NO: 100 - DNA, 32 bp, synthetic construct
  misc_feature 1..32: note = Primer
  source 1..32: mol_type = other DNA
gtgtgagttttgtcgctagcctgggggacctg

SEQ ID NO: 101 - DNA, 32 bp, synthetic construct
  misc_feature 1..32: note = Primer
  source 1..32: mol_type = other DNA
caggtcccccaggctagcgacaaaactcacac

SEQ ID NO: 102 - DNA, 31 bp, synthetic construct
  misc_feature 1..31: note = Primer
  source 1..31: mol_type = other DNA
gcggccgctcatttacccggagacagggaga